SEQUENCE LISTING (1 sequence)

SEQ ID NO: 1 - protein, 12 aa, Artificial Sequence
  Antagonistic polypeptide
FHESWPTFLSPS

(19) United States
(12) Patent Application Publication
(10) Pub. No.: US 2017/0204140 A1
(43) Pub. Date: Jul. 20, 2017

(54) ANTAGONISTIC PEPTIDES

(71) Applicant: Deakin University, Geelong, Victoria (AU)

(72) Inventor: Cenk Suphioglu, Waurn Ponds, Victoria (AU)

(21) Appl. No.: 15/328,309

(22) PCT Filed: Jul. 23, 2015
(86) PCT No.: PCT/AU2015/000432

§ 371 Date: Jan. 23, 2017

(30) Foreign Application Priority Data
Jul. 23, 2014 (AU) 2014902844

(51) Int. Cl. C07K 7/08; C07K 16/38

(52) U.S. Cl. CPC C07K 7/08; C07K 16/38; A61K 38/00

(57) ABSTRACT

The present invention relates to a compositions and methods for treating and/or preventing neurologic disorders such as Alzheimer's disease and disorders associated with an increase of the Th2 immune response such as allergic inflammation.

### FIELD OF THE INVENTION

The present invention relates to a compositions and methods for treating and/or preventing neurologic disorders such as Alzheimer's disease and disorders associated with an increase of the Th2 immune response such as allergic inflammation.

### BACKGROUND OF THE INVENTION

Alzheimer's disease (AD) is a chronic and debilitating degenerative disorder of the brain affecting a significant proportion of the population over the age of 65. The disease is characterized by two main pathological features consisting of senile plaques and neurofibrillary tangles. Specifically, senile plaques are developed from the aggregation of amyloid beta (Aβ), a 40-42 amino acid peptide fragment derived from the amyloid precursor protein (APP) by the sequential action of enzymes. Prior to plaque deposition, these Aβ fragments form toxic oligomers that decrease normal cellular function of neurons in the brain and activate an immune response that leads to apoptosis.

The burden of disease extends worldwide with the most common cause of dementia, Alzheimer's disease, affecting approximately 3% of the population between the ages of 65 to 74. The incidence of disease significantly increases to 50 percent of the world's population over 85 years of age. The number value put on this devastation reached 26.6 million worldwide in 2006 but the toll on life is predicted to only get worse. Long range mathematical models into testing the incidence of the disease predict the number of affected people with Alzheimer's disease will increase to a possible 106 million by 2050.

Atopic disease is rife in the global community, with a doubling in the prevalence of allergy sufferers in the Western world over the past two decades. In 2007, the Australasian Society of Clinical Immunology and Allergy (ASCIA) reported that 4.1 million Australians suffered from an allergic condition. There are many sources of allergens, including dust mites, pollen, air pollutants, insect venom and commercial drugs. Food is also a potent source of allergens; peanut allergy for example poses a particular threat, as fatal anaphylaxis can be brought about by mere traces of peanut and, unlike other food allergies, the condition rarely improves with age. Asthma is also seen as a significant allergic disease; this is a chronic inflammatory disease of the airways that in the developed world represents a major health burden to the community. The quality of life of asthmatics is often compromised by poorer general health, depression and restricted lifestyle. Glucocorticoids (corticosteroids) are the mainstay of long-term asthma management, but are ineffective in preventing either the accelerated loss of lung function or structural changes that occur in the airways due to persistent, chronic inflammation. With a current financial burden in excess of 7 billion dollars per annum and a projected 70% increase in prevalence of allergic disease by the year 2050, there is an urgent need for growth in allergy research to develop new effective therapeutics for the treatment and prevention of allergic disease. A critical step of the allergic response involves the production of allergen-specific IgE antibodies by B cells, as a result of interaction between the cytokines IL-4 or IL-13 and the IL-4 receptor alpha chain (IL-4Rα). These allergen-specific IgE antibodies bind to basophils and mast cells, which on subsequent allergen exposure are cross-linked by the allergen, activating the release of pharmacological mediators of the allergic reaction.

It would be beneficial to have effective therapeutic inhibitors to reduce Aβ production in patients with neurological diseases and disorders, such as AD, and effective therapeutic inhibitors to treating an individual having a disorder associated with an increase of the Th2 immune response, such as an allergic reaction or an allergic inflammation.

The invention provided herein relates to such inhibitors, including their use in a variety of methods.

### SUMMARY OF THE INVENTION

In one aspect the present invention provides an isolated polypeptide that binds specifically to BACE1 and/or IL-4Rα, wherein the polypeptide comprises the amino acid sequence FHESWPTFLSPS or a biologically active derivative thereof.

In one embodiment, the polypeptide binds specifically to BACE1 and inhibits BACE1 enzyme activity. In an embodiment, the polypeptide inhibits endogenous BACE1 enzyme activity, and/or the polypeptide inhibits amyloid-β (Aβ) secretion from cells.

In another embodiment, the polypeptide binds specifically to IL-4Rα and inhibits IL-4Rα binding to IL-13 and/or IL-4. In an embodiment, the polypeptide inhibits JAK-STAT signaling.

In an aspect, the present invention provides an isolated polynucleotide encoding a polypeptide described herein, or a complement thereof.

In an aspect, the present invention provides a vector comprising a polynucleotide described herein.

In an aspect, the present invention provides a host cell comprising a vector described herein.

In an aspect, the present invention provides a method of producing a polypeptide comprising culturing the host cell described herein under conditions in which the polynucleotide is expressed.

In an aspect, the present invention provides an antibody that specifically binds to the polypeptide described herein, or a fragment thereof.

In an aspect, the present invention provides a kit comprising a polypeptide described herein.

In an aspect, the present invention provides a pharmaceutical formulation comprising a polypeptide described herein, and a pharmaceutically acceptable carrier.

In an aspect, the present invention provides a method of treating an individual having a neurological disease or disorder, said method comprising administering to the individual an effective amount of a polypeptide described herein.

In an aspect, the present invention provides a method of reducing amyloid plaques in a patient suffering from, or at risk of contracting, a neurological disease or disorder, said method comprising administering to the individual an effective amount of a polypeptide described herein.

In an aspect, the present invention provides a method of inhibiting amyloid plaque formation in a patient suffering from, or at risk of developing, a neurological disease or disorder, said method comprising administering to the individual an effective amount of a polypeptide described herein. In one embodiment, the neurological disease or disorder is Alzheimer's disease (AD).

In an aspect, the present invention provides a method of reducing amyloid-β (Aβ) protein in a patient comprising administering to the patient an effective amount of a polypeptide described herein.

In one embodiment, the patient is suffering from, or at risk of contracting, a neurological disease or disorder. In another embodiment the neurological disease or disorder is Alzheimer's disease (AD).

In an aspect, the present invention provides a method of treating an individual having a disorder associated with an increase of the Th2 immune response, said method comprising administering to the individual an effective amount of a polypeptide described herein.

In one embodiment, the disorder is associated with an allergic reaction or an allergic inflammation. In another embodiment, the disorder is associated with a mucus production or a mucus secretion. In another embodiment, the disorder is selected from the group consisting of allergic inflammation, allergic asthma, obstructive pulmonary disease, or adult respiratory distress syndrome.

In an aspect, the present invention provides a method of reducing IgE in a patient comprising in a patient comprising administering to the patient an effective amount of a polypeptide described herein. In one embodiment, the patient is suffering from, or at risk of contracting, a disorder associated with an increase of the Th2 immune response. In another embodiment, the disorder is associated with an allergic reaction or an allergic inflammation. In a further embodiment, the disorder is associated with a mucus production or a mucus secretion. In another embodiment, the disorder is selected from the group consisting of allergic inflammation, allergic asthma, obstructive pulmonary disease, or adult respiratory distress syndrome.

In an aspect, the present invention provides a polypeptide as described herein for use as a medicament.

In an aspect, the present invention provides a polypeptide as described herein for use in treating Alzheimer's disease (AD). In one embodiment, the polypeptide as described herein is for use in decreasing and/or inhibiting amyloid-β (Aβ) protein production.

In an aspect, the present invention provides a use of the polypeptide described herein in the manufacture of a medicament. In one embodiment, the medicament is for the treatment of Alzheimer's disease (AD). In another embodiment, the medicament is for reducing and/or inhibiting amyloid-β (Aβ) protein production.

In an aspect, the present invention provides a polypeptide as described herein for use in treating a disorder selected from the group consisting of allergic inflammation, allergic asthma, obstructive pulmonary disease, or adult respiratory distress syndrome.

In one aspect, the present invention provides a polypeptide as described herein for use in decreasing and/or inhibiting JAK-STAT signaling.

In one aspect, the present invention provides a use of the polypeptide as described herein in the manufacture of a medicament. In one embodiment, the medicament is for the treatment of a disorder selected from the group consisting of allergic inflammation, allergic asthma, obstructive pulmonary disease, or adult respiratory distress syndrome.

In one embodiment, the medicament is for reducing and/or inhibiting JAK-STAT signaling.

In an aspect, the present invention provides an isolated polypeptide comprising an amino acid sequence that competes with the polypeptide described herein for binding to BACE1 and/or IL-4Rα.

In an aspect, the present invention provides an isolated polypeptide as described herein, wherein the polypeptide is conjugated or fused to a cytotoxic agent, an amino acid sequence tag that enhances cell entry, or an amino acid sequence of a protein that normally undergoes absorptive mediated transcytosis or receptor mediated transcytosis through the blood-brain-barrier.

In an aspect, the present invention provides an isolated polypeptide as described herein, wherein the polypeptide is formulated for administration to the lung.

### BRIEF DESCRIPTION OF THE DRAWINGS

FIG. 1 shows a biotinylated peptide antagonist binds specifically to BACE1. The bar graph represents the mean absorbance of light of the developed assay, which directly correlates to the amount of biotinylated peptide binding to the target (BACE1) immobilised on a 96-well plate. Different concentrations of peptide were used to test for binding to the target. The negative control did not contain any peptide and represents the background binding of the streptavidin-HRP conjugate, represented by the 0 nM concentration. The 0.1 nM concentration is significantly (*) different to the negative control. The error bars represent the confidence in the mean absorbance values (P<0.05).

FIG. 2 shows a peptide antagonist that binds specifically to BACE1 inhibits BACE1 enzyme activity. The negative control (0 nM) does not contain any peptide and represents the upper limit of the BACE1 enzyme activity by mean fluorescent units on the y-axis. The peptide concentrations of 100 nM and 200 nM show a significant (*) difference to the upper limit when compared with the negative control. The mean fluorescent units are a representation of the cleaved product of APP by BACE1 (P<0.0 5). BACE1 peptide antagonist demonstrates more than 40% inhibition of BACE1 activity (indicated by vertical arrows).

FIG. 3 shows a peptide antagonist that binds specifically to BACE1 inhibits BACE1 enzyme activity in cultured human neuronal cells. The M17 cell lysate was subjected to BACE1 enzyme activity assay and was read on a fluorometer in duplicate with one experimental group of a positive control, without the peptide inhibitor (0 nM), and an experimental group, with an added 200 nM concentration of the peptide inhibitor. The cell lysate sample is displayed on the x-axis, with and without the peptide inhibitor. The mean fluorescent units are a representation of the cleaved product of APP by BACE1 (P<0.0 5). This indicates the production of BACE1 in cultured human neuronal cells and the usefulness of this cell line. Moreover, BACE1 peptide antagonist demonstrates more than 50% inhibition of endogenous BACE1 activity (indicated by the vertical arrow).

FIG. 4 shows the effect of DHA on protein expression in M17 human neuronal cells. Proteome expression of M17 cells grown in the presence of zinc (final concentration of 5 μM) and no DHA (a) or with 10 μg/mL DHA (b). Gels were stained with SYPRO Ruby staining. Protein spots of significant difference (Arrows) were subjected to MS analysis (S1, S2). (c) Protein spots S1 and S2 were excised from each gel by automated robotic cutter and subjected to trypsin digest followed by MS analysis and submission of peptide fingerprints to Homo sapiens National Center for Biotechnology Information database searches. Proteins were identified via their peptide mass fingerprint and deduced amino acid sequence determined by single MS and tandem MS/MS, respectively. Protein identity was only reported for samples that gave a significant (P<0.05) molecular weight search score. pI, isoelectric point; Mr, molecular mass.

FIG. 5 shows an inhibitor of BACE1 inhibits Aβ secretion in a dose dependent manner. This figure shows a dose effect of the commercial BACE1 inhibitor, Compound IV (Merck Calbiochem) on Aβ release from a cellular model. Cells were treated for 5 hrs with compound concentrations ranging from 1 nM to 1 μM. Aβ secreted in the cell media was quantified using a sandwich ELISA. Data are calculated relative to Aβ secreted from control cells treated with DMSO (vehicle) alone.

FIG. 6 shows a biotinylated peptide antagonist binds specifically to IL-4Rα. A biotinylated version of the peptide antagonist could also interact with IL-4Rα in a dose-dependent manner, compared with a relevant negative control.

FIG. 7 shows a biotinylated peptide antagonist that binds specifically to IL-4Rα inhibits the binding of IL-13 and IL-4 to IL-4Rα. In order to establish whether this synthetic peptide showed any inhibition of IL-4/IL-13 binding to IL-4Rα, we performed inhibition ELISA. Pre-incubation of IL-4Rα with nM amounts of the peptide reduced IL-13 binding to IL-4Rα by 50% and IL-4 binding by 25%, suggesting that this peptide antagonist holds great promise as a potent inhibitor of IL-4/IL-13 interaction with IL-4Rα.

FIG. 8 shows the biomolecular interaction between IL-4 and IL-4Rα. IL-4Rα was successfully immobilised on a CM-5 sensor chip and a single kinetic result for IL-4 interacting with IL4Rα is shown, demonstrating an exceptional fit for 1:1 binding and fast association rate for IL-4.

FIG. 9 shows a peptide antagonist that binds specifically to IL-4Rα inhibits JAK-STAT signaling. This data shows a dose-dependent inhibition of the JAK-STAT6 signaling in STAT6 signaling in HEK-Blue cells, with up to 59% inhibition at 225 μM. The peptide antagonist itself (without IL-4) did not trigger a non-specific JAK-STAT6 signaling, suggesting that this peptide antagonist has specificity and efficacy. Positive Control (PC) included cells treated with IL-4 cytokine alone. Control 1 (C1) included cells incubated with peptide antagonist only and Control 2 (C2) included cells without any treatment. The results represent the mean of 3 replicates within an experiment and the error bars show standard deviation. P<0.05* denotes significance of optical density (OD) readings compared to positive control (PC).

FIG. 10 shows studies using the sheep asthma model highlight the presence of IL-4 and IL-13 in BAL fluid following airway allergen challenge suggesting involvement of these key Th2 cytokines in the airway inflammation (recruitment of activated T cells, eosinophilia and IgE responses) typically seen in this animal model system. Shown is an ELISA of IL-4 and IL-13 levels in BAL fluid collected from lung segments 6, 24, and 48 h post challenge with saline or HDM, compared to baseline levels (0 h pre-challenge).

FIG. 11 shows a schematic for in vivo sheep experiments.

FIG. 12 shows the percent inhibition of HEK-Blue cells with a peptide antagonist that binds specifically to IL-4Rα. The percentage values represented here are a reflection of treatments used in FIG. 9 (75 μM, 150 μM and 225 μM). This Figure shows incremental inhibition of IL-4Rα signaling from HEK-Blue cells as the concentrations of the peptide antagonist that binds specifically to IL-4Rα concentrations are increased. Positive control is denoted at 0%, which is used as a referral point for the standard value at which inhibition begins.

### DETAILED DESCRIPTION

Pathologically, senile plaque formation in Alzheimer's disease begins with the proteolytic processing of the amyloid precursor protein (APP). The membrane bound protein is processed by multiple proteolytic cuts leading to the production of 38-43 amino acid peptide derivatives known as amyloid beta (Aβ). The longer peptides are insoluble and form toxic aggregated species. The proteolytic processing of APP mainly involves the three enzymes α, β and γ-secretase. The extracellular domain of the protein can be alternatively processed by α or β-secretase (BACE1) to release APP ectodomain fragments, sAPPα and sAPPβ, from cellular membranes. γ-secretase cuts the remaining, membrane-bound C-99 fragment produced by BACE1 cleavage whilst in the intramembrane space, releasing the amyloid precursor protein intracellular domain (AICD) and Aβ. Cleavage of APP by α-secretase is non-amyloidogenic and generates the shorter, non-amyloidogenic C-terminal fragment C83.

The main mechanisms relating to the progression of AD are instigated by the onset of amyloid plaques and neurofibrillary tangles. Therapeutic relief of drugs like donepezil, galantamine and memantine are required to optimize the productivity of the brain whilst the disease progresses and cannot translate to a definite cure. Current novel therapeutic approaches target the amyloid cascade, in particular the secretase enzymes that produce Aβ. Despite intensive research in the area of the production of Aβ, research has failed so far to produce a drug amenable to the clinic. The use of gamma-secretase inhibitors for AD treatment is unlikely to provide a viable option as this enzyme is required for the signaling of many cellular receptors and such inhibitors are now trialed in cancer treatment. Without wishing to be bound by theory, the present inventors consider BACE1 appears to be a safer target as it acts on few receptors other than APP. However, designing BACE1 inhibitors with in vivo efficacy has so far remained a challenge in the field.

Modern drug discovery approaches to BACE1 inhibition include high-throughput screening (HTS), fragment-based drug discovery (FBDD) and substrate-based aspartyl protease inhibitors, however, none of these methods have yet been successful in therapeutic trials.

BACE1 belongs to the class of aspartyl proteases, like renin and the HIV protease for which successful inhibition programs have been developed. Although BACE1 displays significant homology to the other enzymes of the pepsin family, its catalytic site is more open, less hydrophobic, and larger than that of its counterparts.

The HTS method has been substituted for the FBDD approach because it uses smaller and more specific compounds. The screening of a fragment library is more appealing because a higher “hit” ratio is produced and the options show favourable drug properties. The main problem with the “hit” compounds is again the low potency and selectivity. Often, fragments that showed promise were too small to be effective and have not provided any real inhibition with the effectiveness required for therapeutic trials.

By using phage display technology, the present inventors have successfully identified synthetic peptide antagonists of BACE1.

By using phage display technology, the present inventors have successfully identified a 12-mer synthetic peptide antagonist of BACE1. In addition, the synthetic peptide successfully inhibited BACE1 activity in cleavage of APP.

The present inventors have demonstrated that peptides of the present invention, due to their binding of BACE1, inhibits BACE1 enzyme activity, in a manner to bring about a therapeutic response, inhibiting amyloid-β secretion from cells.

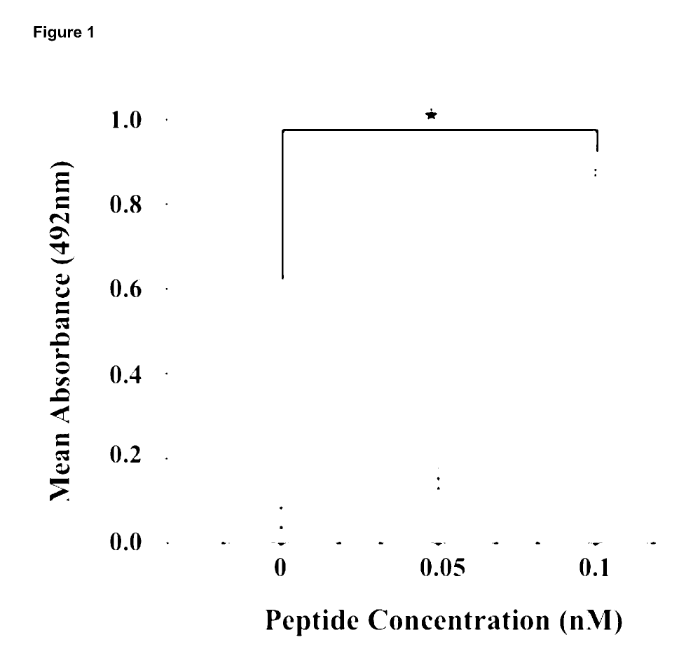

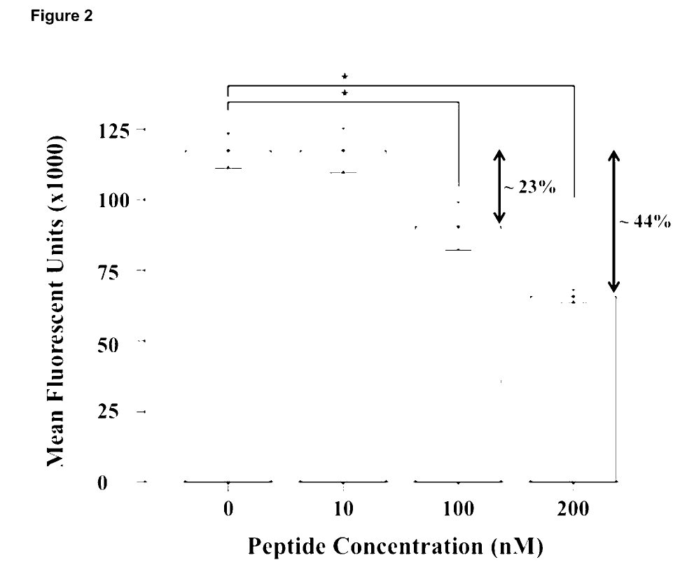

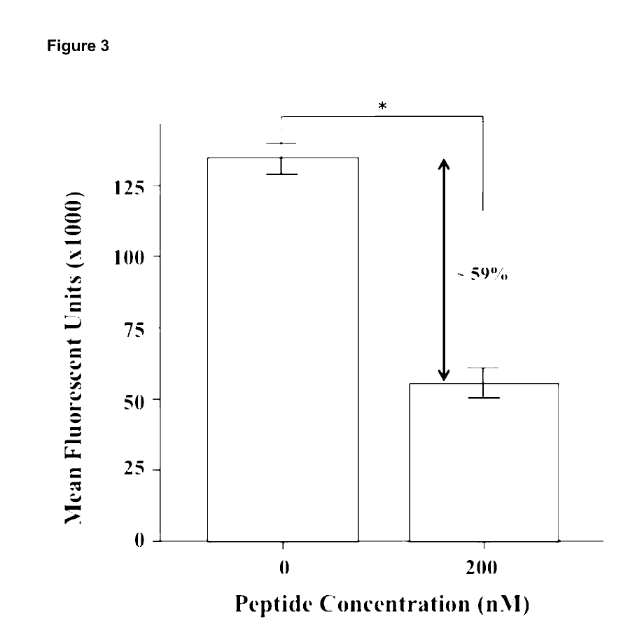

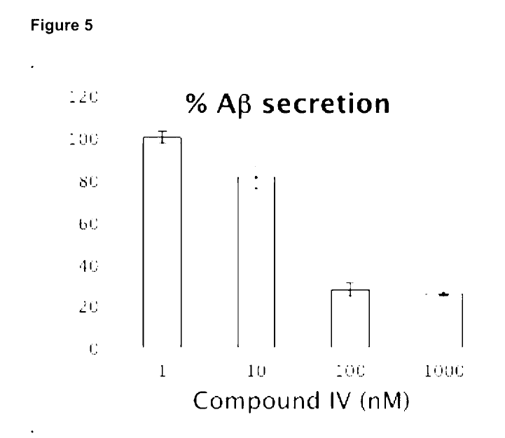

For example, the present inventors have shown an isolated polypeptide that binds specifically to BACE1. FIG. 1 shows the polypeptide FHESWPTFLSPS binds specifically to BACE1, and binding of the peptide is dose dependent. Importantly, FIG. 2 demonstrates the polypeptide FHESWPTFLSPS which binds specifically to BACE1, inhibits a BACE1 enzyme activity, reducing the amount of cleaved product of APP. FIG. 3 demonstrates the polypeptide FHESWPTFLSPS which binds specifically to BACE1, inhibits an endogenous BACE1 enzyme activity in neuronal cells, reducing the amount of cleaved product of APP. The present inventors have also demonstrated (e.g. FIG. 5) that a BACE1 inhibitor can inhibit Aβ secreted from the CHO-APP cell line described herein.

Therefore, in one aspect, the present invention provides an isolated polypeptide that binds specifically to BACE1, wherein the polypeptide comprises the amino acid sequence FHESWPTFLSPS or a derivative thereof.

The present inventors have also demonstrated that peptides of the present invention, due to their binding of the IL-4 receptor alpha chain, interfere with the interaction of the receptors' cognate ligands, i.e., the cytokine(s) IL-4 and/or IL-13, in a manner to bring about a therapeutic response, inhibiting signaling via the JAK-STAT pathway.

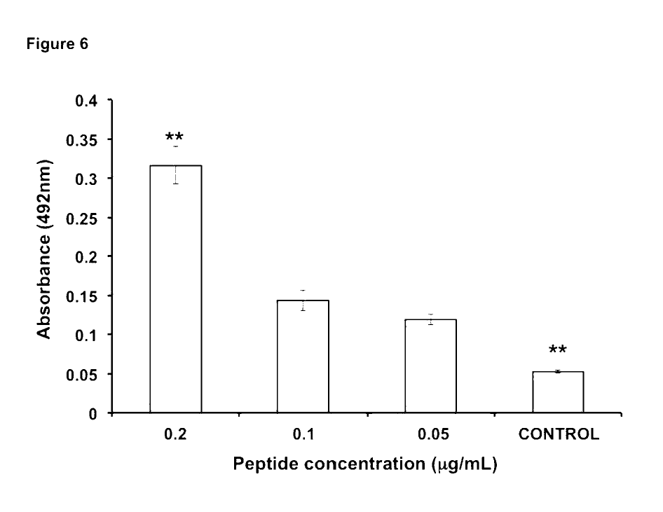

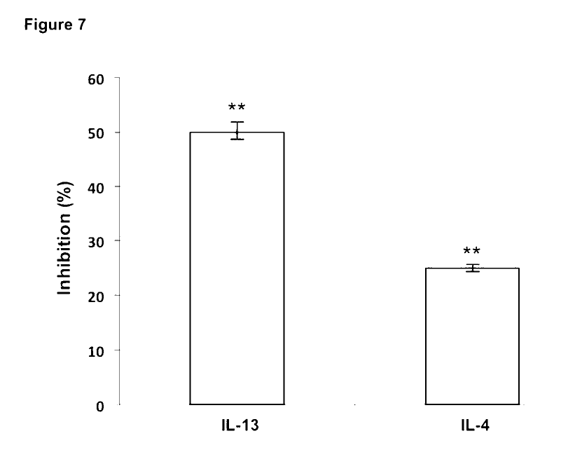

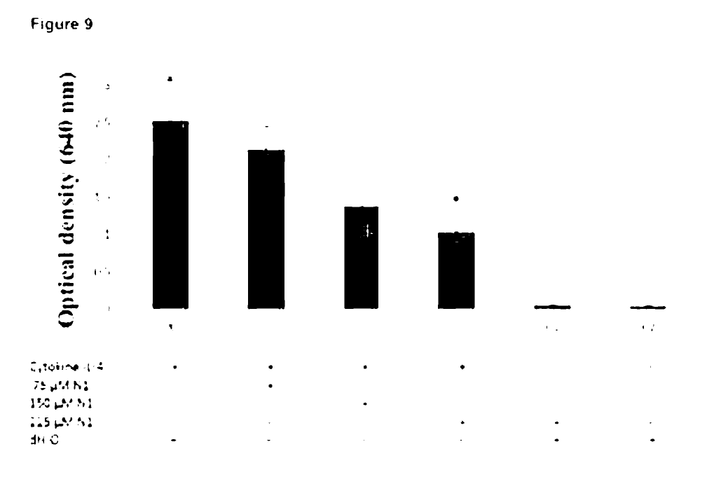

For example, the present inventors have shown an isolated polypeptide that binds specifically to IL-4Rα. FIG. 6 shows the polypeptide FHESWPTFLSPS binds specifically to IL-4Rα, and binding of the peptide is dose dependent. Importantly, FIG. 7 demonstrates the polypeptide FHESWPTFLSPS which binds specifically to IL-4Rα, inhibits the interaction of biologically active human IL-4Rα with IL-4 and IL-13. FIG. 9 demonstrates the polypeptide FHESWPTFLSPS which binds specifically to IL-4Rα, inhibits JAK-STAT signaling.

Accordingly, in one aspect, the present invention provides an isolated polypeptide that binds specifically to BACE1 and/or IL-4Rα, wherein the polypeptide comprises the amino acid sequence FHESWPTFLSPS (SEQ ID NO: 1) or a biologically active derivative thereof.

“Isolated,” when referred to a molecule, refers to a molecule that has been identified and separated and/or recovered from a component of its natural environment. Contaminant components of its natural environment are materials that interfere with diagnostic or therapeutic use.

The peptides of the present invention can be isolated and/or purified (or substantially isolated and/or substantially purified). The peptides can be isolated from other peptides as a result of solid phase protein synthesis, for example. Alternatively, the peptides can be substantially isolated from other proteins after cell lysis from recombinant production. Standard methods of protein purification (e.g., HPLC) can be employed to substantially purify the inventive peptides. Thus, a preparation of the peptide according to the present invention preferably is at least 90% (by weight) free of other peptides and/or contaminants, and more preferably is at least about 95% (by weight) free of other peptides and/or contaminants (such as at least about 97% or 98% (by weight) free of other peptides and/or contaminants).

The term “peptide” generally refers to a contiguous and relatively short sequence of amino acids linked by peptidyl bonds. Typically, but not necessarily, a peptide has a length of about 2 to 50 amino acids, 4-40 amino acids or 10-30 amino acids. Although the term “polypeptide” generally refers to longer forms of a peptide, the two terms can be and are used interchangeably in some contexts herein.

The peptides of the present invention may be produced by any suitable method known in the art, such as chemical synthesis and/or recombinant DNA technology. For example, the inventive peptide can be synthesized using solid phase peptide synthesis techniques (e.g., Fmoc). Alternatively, the peptide can be synthesized using recombinant DNA technology (e.g., using bacterial or eukaryotic expression systems). A nucleotide sequence encoding a polypeptide of the invention may be constructed by isolating or synthesizing a nucleotide sequence encoding the parent peptide and then changing the nucleotide sequence so as to effect introduction (i.e. insertion or substitution) or removal (i.e. deletion or substitution) of the relevant amino acid residue(s). Methods for solid state protein synthesis and recombinant protein synthesis are well-known in the art.

A variant of a peptide may be naturally occurring or it may be a variant that is not known to occur naturally. Non-naturally occurring variants of peptides may be made by direct synthesis, or alternatively, mutations can be introduced randomly along all or part of a peptide of this invention, such as by saturation mutagenesis or site-directed mutagenesis in accordance with conventional methods. Independent of the method of production, the resultant variants can be screened for the ability of binding specifically to BACE1 and/or IL-4Rα.

The term “peptide” includes not only molecules in which amino acid residues are joined by peptide (—CO—NH—) linkages but also molecules in which the peptide bond is reversed.

A retro-inverso (R-I) version of an isolated polypeptide as described herein is included herein with inversion of chirality (L→D amino acids) and also reversal of sequence (carboxyl→amino) using all D-amino acid monomers and retroversion of the amino acid sequence: amino→carboxyl becoming carboxyl→amino (Chorev M, The partial retro-inverso modification: a road traveled together. Biopolymers. 2005; 80:67-84). R-I peptide approach provides a general method for generation of metabolically stable mimics of biologically active peptides for diagnostics and therapeutics.

The retro-inverso (R-I) peptide synthetic modification (Pallai P V, Richman S, Struthers R S, Goodman M. Approaches to the synthesis of retro-inverso peptides. Int J Pept Protein Res. 1983; 21:84-92) may involve both inversion of amino acid a-carbon chirality and reversal of peptide bonds (i.e., reversal of primary amino acid sequence), with the goal of increasing peptide stability while preserving or reconstituting side-chain orientations.

R-I peptides may be made using methods known in the art. For example, this approach can involve making pseudopeptides containing changes involving the backbone, and not the orientation of side chains. Retro-inverse peptides, which contain NH—CO bonds instead of CO—NH peptide bonds, are much more resistant to proteolysis. Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH2-″—NH(Me) or —N(Me)2).

The term “biologically active” as used herein includes the ability of the polypeptide to bind specifically to BACE1 and/or IL-4Rα, inhibit the interaction of biologically active human IL-4Rα with IL-4 and IL-13, inhibits JAK-STAT signaling, inhibit BACE1 enzyme activity, and/or inhibit amyloid-β secretion from cells.

As used herein, the term “binds specifically” refers to the ability of a peptide antibody bind to an antigen with a Kd of at least about 1×10⁻³ M, 1×10⁻⁴ M, 1×10⁻⁵ M, 1×10⁻⁶ M, 1×10⁻⁷ M, 1×10⁻⁸ M, 1×10⁻³ M, 1×10⁻¹³ M, 1×10⁻¹¹ M, 1×10⁻¹² M, or more. The term also encompasses refers to the ability of peptide to bind to an antigen with an affinity that is at least two-fold greater than its affinity for a nonspecific antigen. It shall be understood, however, that a peptide is capable of specifically binding to two or more antigens which are related in sequence, and in the present case, bind specifically to BACE1 and/or IL-4Rα.

Binding assays can be performed in a variety of formats, including protein-protein binding assays, biochemical screening assays, immunoassays, and cell-based assays, which are well characterized in the art.

Assays for polypeptides that bind BACE1 and/or IL-4Rα are common in that they call for contacting the candidate modulator with BACE1 and/or IL-4Rα (or equivalent thereof) and/or binding ligand that is involved in the binding interaction of BACE1 and/or IL-4Rα and the binding ligand, under conditions and for a time sufficient to allow these two components to interact.

In binding assays, the interaction is binding and the complex formed can be isolated or detected in the reaction mixture. In a particular embodiment, a candidate substance or molecule is immobilized on a solid phase, e.g., on a microtiter plate, by covalent or non-covalent attachments. Non-covalent attachment generally is accomplished by coating the solid surface with a solution of the substance/molecule and drying. Alternatively, an immobilized affinity molecule, such as an antibody, e.g., a monoclonal antibody, specific for the substance/molecule to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

Candidate substance molecules (e.g. polypeptides) can be generated by combinatorial libraries and/or mutations of known binders based on information described herein, in particular information relating to contributions and importance to BACE1 and/or IL-4Rα-ligand binding interactions of individual residues and moieties within a ligand or BACE1 and/or IL-4Rα sequence itself.

Compounds that interfere with the interaction of BACE1 and/or IL-4Rα and binding ligand can be tested as follows: usually a reaction mixture is prepared containing BACE1 and/or IL-4Rα and a ligand under conditions and for a time allowing for the interaction and binding of the two molecules. To test the ability of a candidate compound to inhibit the binding interaction, the reaction is run in the absence and in the presence of the test compound. In addition, a control compound may be added to a third reaction mixture, to serve as positive control. The binding (complex formation) between the test compound and BACE1 and/or IL-4Rα and/or binding ligand present in the mixture is monitored, as described hereinabove. The formation of a complex in the control reaction(s) but not in the reaction mixture containing the test compound indicates that the test compound interferes with the interaction of BACE1 and/or IL-4Rα and binding ligand.

Forming a complex of a binding polypeptide and target (e.g. a polypeptide that binds BACE1 and/or IL-4α) facilitates separation of the complexed from the uncomplexed forms thereof and from impurities. Binding polypeptide:ligand complexes can be formed in solution or where one of the binding partners is bound to an insoluble support. The complex can be separated from a solution, for example using column chromatography, and can be separated while bound to a solid support by filtration, centrifugation, etc. using well-known techniques. Binding BACE1 and/or IL-4α a to a solid support facilitates high throughput assays.

Test polypeptides can be screened for the ability to modulate (e.g., inhibit) the interaction of a binding polypeptide with BACE1 and/or IL-4α in the presence and absence of a candidate binding polypeptide, and screening can be accomplished in any suitable vessel, such as microtiter plates, test tubes, and microcentrifuge tubes. Fusion proteins can also be prepared to facilitate testing or separation, where the fusion protein contains an additional domain that allows one or both of the proteins to be bound to a matrix. For example, GST-IL-4α-binding peptide fusion proteins or GST-IL-4α proteins, or GST-BACE1-binding peptide fusion proteins or GST-BACE1 proteins can be adsorbed onto glutathione sepharose beads (SIGMA Chemical, St. Louis, Mo.) or glutathione derivatized microtiter plates that are then combined with the test compound, and the mixture is incubated under conditions allowing complex formation (e.g., at physiological conditions of salt and pH). Following incubation, the beads or microtiter plate wells are washed to remove any unbound components, the matrix immobilized in the case of beads, and the complex determined either directly or indirectly. Alternatively, the complexes can be dissociated from the matrix, and the level of binding or activity determined using standard techniques.

Other fusion polypeptide techniques for immobilizing proteins on matrices can also be used in screening assays. Either a binding polypeptide or BACE1 or IL-4α can be immobilized using biotin-avidin or biotin-streptavidin systems. Biotinylation can be accomplished using many reagents, such as biotin-N-hydroxy-succinimide (NHS; PIERCE Chemicals, Rockford, 111.), and immobilized in wells of streptavidin coated 96 well plates (PIERCE Chemical). Alternatively, antibodies reactive with BACE1 or IL-4α binding polypeptides or BACE1 or IL-4α but which do not interfere with binding of a binding polypeptide to its target molecule can be derivatized to the wells of the plate, and unbound BACE1 or IL-4α or binding polypeptide trapped in the wells by antibody conjugation. Methods for detecting such complexes, in addition to those described for the GST-immobilized complexes, include immunodetection of complexes using antibodies reactive with the binder peptides, IL-4α or BACE1.

To assess the binding affinities of a polypeptide, protein or other BACE1 or IL-4α ligand, competition binding assays may be used, where the ability of the ligand to bind BACE1 and/or IL-4α (and the binding affinity, if desired) is assessed and compared to that of a compound known to bind BACE1 and/or IL-4α, for example, a peptidomimetic inhibitor of BACE1 and/or IL-4α, an anti-IL-4α or anti-BACE1 antibody, or a high-affinity binding polypeptide determined e.g. such as those as described herein.

Many methods are known and can be used to identify the binding affinities of binding molecules (e.g. polypeptides, proteins, small molecules, etc.); for example, binding affinities can be determined as IC₅₀ values using competition ELISAs. The IC₅₀ value is defined as the concentration of binder (e.g. polypeptide) that blocks 50% of BACE1 and/or IL-4α binding to a ligand. For example, in solid phase assays, assay plates may be prepared by coating microwell plates (preferably treated to efficiently adsorb protein) with neutravidin, avidin or streptavidin. Non-specific binding sites are then blocked through addition of a solution of bovine serum albumin (BSA) or other proteins (for example, nonfat milk) and then washed, preferably with a buffer containing a detergent, such as Tween-20. A biotinylated BACE1 or IL-4α binding polypeptide (for example, as described in the Examples) is prepared and bound to the plate. Serial dilutions of the molecule to be tested with BACE1 or IL-4α are prepared and contacted with the bound binding polypeptide. The plate coated with the immobilized binder is washed before adding each binding reaction to the wells and briefly incubated. After further washing, the binding reactions are detected, often with an antibody recognizing the non-BACE1 or not IL-4α fusion partner and a labeled (such as horseradish peroxidase (HRP), alkaline phosphatase (AP), or a fluorescent tag such as fluorescein) secondary antibody recognizing the primary antibody. The plates are then developed with the appropriate substrate (depending on the label) and the signal quantified, such as using a spectrophotometric plate reader. The absorption signal may be fit to a binding curve using a least squares fit. Thus the ability of the various molecules to inhibit BACE1 or IL-4α from binding a known BACE1 or IL-4α binding molecule can be measured.

Importantly, the present inventors have shown that the peptides of the present invention bind specifically to BACE1 and inhibit BACE1 enzyme activity.

Therefore, in one embodiment, the polypeptide binds specifically to BACE1 and inhibits BACE1 enzyme activity. In another embodiment, the polypeptide inhibits endogenous BACE1 enzyme activity, and/or the polypeptide inhibits amyloid-β (Aβ) secretion from cells.

Determination of the ability of a candidate peptide or polypeptide of the invention (such as a peptide comprising the amino acid sequence of a binding peptide disclosed herein) to modulate BACE1 activity can be performed by testing the modulatory capability of the substance/molecule in in vitro or in vivo assays. Modulatory capability may include, e.g., inhibition or reduction of BACE 1 aspartyl protease activity; or inhibition or reduction in APP cleavage by BACE1; or inhibition or reduction in Aβ production.

In certain embodiments, a peptide/polypeptide of the invention, such as a peptide comprising the amino acid sequence of a binder peptide disclosed herein, is tested for such biological activity, for example, as described in detail in Example 2. For example, BACE1 protease activity can be tested in a homogeneous time-resolved fluorescence HTRF assay or a microfluidic capillary electrophoretic (MCE) assay, using synthetic substrate peptides.

In addition, BACE1 protease activity can be tested in vivo in cell lines which express BACE1 substrates such as APP, such as those described herein, or in transgenic mice which express BACE1 substrates, such as human APP.

Additionally, BACE1 protease activity can be tested in animal models. For example, animal models of various neurological diseases and disorders, and associated techniques for examining the pathological processes associated with these models, are readily available in the art. Animal models of various neurological disorders include both non-recombinant and recombinant (transgenic) animals. Non-recombinant animal models include, for example, rodent, e.g., murine models. Such models can be generated by introducing cells into syngeneic mice using standard techniques, e.g. subcutaneous injection, tail vein injection, spleen implantation, intraperitoneal implantation, and implantation under the renal capsule. In vivo models include models of neurodegenerative diseases, such as mouse models of Alzheimer's disease. The various assays may be conducted in known in vitro or in vivo assay formats, as known in the art and described in the literature. Various such animal models are also available from commercial vendors such as the Jackson Laboratory.

As used herein, the terms “inhibits”, “reduce” and “decrease” refer to a decrease in any activity of the molecule targeted by the peptides described herein. For example, a decrease in any activity of BACE1 and/or IL-4Rα, including, but not limited to, the activities described herein. The term includes decreasing any activity of the molecule by directly binding the molecule.

In one embodiment, the isolated polypeptide directly interacts with at least one specific BACE1 residue.

The term “BACE1” as used herein, refers to any native beta-secretase 1 (also called β-site amyloid precursor protein cleaving enzyme 1, membrane-associated aspartic protease 2, memapsin 2, aspartyl protease 2 or Asp2) from any vertebrate source, including mammals such as primates (e.g. humans) and rodents (e.g. mice and rats), unless otherwise indicated. The term encompasses “full-length”, unprocessed BACE1 as well as any form of BACE1 that results from processing in the cell. The term also encompasses naturally occurring variants of BACE1, e.g. splice variants or allelic variants. The amino acid sequence of an exemplary BACE1 polypeptide is reported in Vassar et al. Science 286:735-741 (1999), which is incorporated herein by reference in its entirety.

The peptides of the present invention may be modified in other ways, for example to increase or decrease the peptide's half-life in vivo. Thus peptoid analogues, D-amino acid derivatives, and peptide-peptoid hybrids may be used, as discussed above. Therefore, a further embodiment of the polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration. Other post-translational modifications may be used, provided they do not significantly reduce binding of the peptide to BACE 1 and/or IL-4Rα.

BACE1 as used herein may be produced as a purified protein or protein fragment (e.g., the extracellular domain, residues 22-457, residues 43-453 or residues 57-453 of BACE1) or as a fusion polypeptide using conventional synthetic or recombinant techniques. Fusion polypeptides are useful in phage display wherein BACE1 is the target for binding, in expression studies, cell-localization, bioassays, ELISAs (including binding competition assays), etc. A BACE1 “chimeric protein” or “fusion protein” comprises BACE1 fused to an unrelated polypeptide. A BACE1 fusion protein may include any portion up to the entire sequence of BACE1, including any number of the biologically active portions. The fusion protein can then be purified according to known methods using affinity chromatography and a capture reagent that binds to the non-BACE1 polypeptide. BACE1 may be fused to an affinity sequence, e.g. the C-terminus of the GST (glutathione S-transferase) sequences. Such fusion proteins facilitate the purification of the recombinant BACE1 using, e.g., glutathione bound to a solid support and/or attachment to solid support (e.g., a matrix for peptide screening/selection/biopanning).

Alanine scanning of a binding polypeptide sequence can be used to determine the relative contribution of each residue in the peptide to binding and/or inhibition. To determine the critical residues in a polypeptide, residues are substituted with a single amino acid, typically an alanine residue, and the effect on binding and activity (e.g. BACE1 enzyme activity and/or IL-4α activity) is assessed. See the Examples.

Truncation of a binding polypeptide binding peptide can elucidate not only binding critical residues, but also determine the minimal length of peptide to achieve binding. In some cases, truncation will reveal a ligand that binds more tightly than the native ligand; such a peptide is useful to modulate BACE1 enzyme activity and/or IL-4α activity.

Preferably, a series of binding polypeptide truncations are prepared. One series will truncate the amino terminal amino acids sequentially; in another series, the truncations will begin at the carboxy terminus. As in the case for alanine scanning, the peptides may be synthesized in vitro or prepared by recombinant methods.

The term “BACE1 enzyme activity” as used herein, refers to any BACE1 enzyme activity, including inhibition or reduction of BACE1 aspartyl protease activity; or inhibition or reduction in APP cleavage by BACE1; or inhibition or reduction in Aβ production.

The term endogenous enzyme activity as used herein refers to any enzyme activity in cells or derived from cells into which exogenous nucleic acid has not been introduced.

The term “amyloid-β” (Aβ) as used herein refers to a proteolytic product of the precursor protein, beta amyloid precursor protein (β-APP or APP). APP is a type-I trans-membrane protein which is sequentially cleaved by two proteases, a β- and γ-secretase. The β-secretase, known as β-site amyloid precursor protein cleaving enzyme 1 (BACE1) described herein, first cleaves APP to expose the N-terminus of Aβ, thereby producing a membrane bound fragment known as C99. The γ-secretase then is able to cleave C99 to produce the mature Aβ polypeptide. Aβ is produced with heterogenous C termini ranging in length from 38 amino acids to 43 amino acids. The 42 amino acid form of Aβ (Aβ42) is the fibrillogenic form of Aβ and is over produced in patients with Down's syndrome and has been suggested to play a role in the early pathogenesis of AD.

The terms “amino acid” and “residue” are used interchangeably herein. A “region” of a polypeptide is a contiguous sequence of 2 or more amino acids. In other embodiments, a region is at least about any of 3, 5, 10, or 12 contiguous amino acids.

The terms “standard” and “natural” as applied to peptides herein refer to peptides constructed only from the standard naturally-occurring amino acids: alanine (Ala, A), cysteine (Cys, C), aspartate (Asp, D), glutamate (Glu, E), phenylalanine (Phe, F), glycine (Gly, G), histidine (His, H), isoleucine (He, I), lysine (Lys, K), leucine (Leu, L), methionine (Met, M), asparagine (Asn, N), proline (Pro, P), glutamine (Gin, Q), arginine (Arg, R), serine (Ser, S), threonine (Thr, T), valine (Val, V), tryptophan (Trp, W), and tyrosine (Tyr, Y).

In one embodiment, the isolated peptide has the amino acid sequence FHESWPTFLSPS or Phe-His-Glu-Ser-Trp-Pro-Thr-Phe-Leu-Ser-Pro-Ser in three-letter amino acid code.

The amino acid residues of the peptide can be “L-form” amino acid residues, “D” amino acid residues, or a combination thereof. L-, D-, or β-amino acid versions of the peptide sequence as well as retro, inverso, and retro-inverso isoforms are included, “β-peptides” are comprised of “β amino acids”, which have their amino group bonded to the β carbon rather than the a-carbon as in the 20 standard biological amino acids.

The invention also provides a mutant or variant peptide any of which residues may be changed from the corresponding residues of these peptides, while still encoding a peptide that maintains inhibitory activity, for example, truncated, mutant or variant peptides generated using the methods of the Examples.

In one embodiment, a variant of a binder peptide/polypeptide has at least 50%, 60%, 70%, 80%, 90%, 95%, 98%, 99% amino acid sequence identity with the sequence of a reference binder peptide/polypeptide. Preferably, the variant exhibits substantially the same or greater binding affinity than the reference binder peptide/polypeptide, e.g., at least 0.75×, 0.8×, 0.9×, 1×, 1.25× or 1.5× the binding affinity of the reference binder peptide/polypeptide, based on an accepted binding assay quantitation unit/metric. In general, variants of the invention include variants in which residues at a particular position in the sequence have been substituted by other amino acids, and further includes the possibility of inserting an additional residue or residues between two residues of the binder peptide/polypeptide as well as the possibility of deleting one or more residues from the binder peptide/polypeptide or adding one or more residues to the parent sequence. Any amino acid substitution, insertion, or deletion is encompassed by the invention. In certain circumstances, the substitution is a conservative substitution.

“Percent (%) amino acid sequence identity” is defined as the percentage of amino acid residues that are identical with amino acid residues in a reference (parent) polypeptide sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as: % amino acid sequence identity=X/Y′100 where X is the number of amino acid residues scored as identical matches by the sequence alignment program's or algorithm's alignment of A and B and Y is the total number of amino acid residues in B. If the length of amino acid sequence A is not equal to the length of amino acid sequence B, the % amino acid sequence identity of A to B will not equal the % amino acid sequence identity of B to A.

“C-terminal region”, “C-terminal sequence”, and variations thereof, as used herein, refer to an amino acid sequence that is located at or in close proximity to the C-terminal end of a polypeptide. Generally, the sequence includes an amino acid that has a free carboxyl group. In one embodiment, a C-terminal region or sequence refers to a region of a polypeptide that includes the about 1-15 residues located closest to the C terminus of the polypeptide.

“N-terminal region”, “N-terminal sequence”, and variations thereof, as used herein, refer to an amino acid sequence that is located at or in close proximity to the N-terminal end of a polypeptide. Generally, the sequence includes an amino acid that has a free amino group. In one embodiment, a N-terminal region or sequence refers to a region of a polypeptide that includes the about 1-15 residues located closest to the N terminus of the polypeptide.

“Internal region”, “internal sequence”, and variations thereof, as used herein, refer to an amino acid sequence that is located within a polypeptide and is flanked on both its N- and C-termini by one or more amino acids that are not part of the sequence. Generally, the sequence does not include an amino acid with either a free carboxyl or amino group. In one embodiment, an internal region or sequence refers to a region of a polypeptide that includes the about 1-15 residues located within a polypeptide, wherein the region does not include either the C-terminal or N-terminal amino acid.

The term “derivative” as used in the present disclosure relates to derivatives of a protein or peptide (e.g. polypeptide that binds BACE1 or IL-4Rα of the disclosure) that comprise modifications of the amino acid sequence, for example by substitution, deletion, insertion or chemical modification. Preferably, such modifications do not reduce the functionality of the protein or polypeptide. Such variants include proteins, wherein one or more amino acids have been replaced by their respective D-stereoisomers or by amino acids other than the naturally occurring 20 amino acids, such as, for example, ornithine, hydroxyproline, citrulline, homoserine, hydroxylysine, norvaline. However, such substitutions may also be conservative, i.e. an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan. The term also includes fragments of polypeptides of the disclosure relates, such as proteins or peptides derived from full-length polypeptides described herein that are N-terminally and/or C-terminally shortened, i.e. lacking at least one of the N-terminal and/or C-terminal amino acids, such as those derived from truncation studies derived herein.

“Affinity” refers to the strength of the sum total of non-covalent interactions between a single binding site of a molecule and its binding partner. Unless indicated otherwise, as used herein, “binding affinity” refers to intrinsic binding affinity which reflects a 1:1 interaction between members of a binding pair. The affinity of a molecule X for its partner Y can generally be represented by the dissociation constant (Kd). Affinity can be measured by common methods known in the art, including those described herein. Specific illustrative and exemplary embodiments for measuring binding affinity are described herein.

An “active” polypeptide, or fragments thereof, retains a biological activity of the native or naturally-occurring counterpart of the active polypeptide. Biological activity refers to a function mediated by the native or naturally-occurring counterpart of the active polypeptide. For example, binding or protein-protein interaction constitutes a biological activity.

The present inventors have shown the polypeptide binds specifically to BACE1 and inhibits BACE1 enzyme activity. Importantly, the present inventors have shown the polypeptide inhibits endogenous BACE1 enzyme activity. The assays described herein demonstrate inhibition of amyloid-β (Aβ) secretion from cells.

APP is a type-I trans-membrane protein which is sequentially cleaved by two proteases, a β- and γ-secretase. BACE1 cleaves APP to expose the N-terminus of Aβ, producing a membrane bound fragment known as C99. The γ-secretase then cleaves C99 to produce the mature Aβ polypeptide. Therefore, the term “secretion” as used herein includes the production of Aβ, which is the proteolytic product of the precursor protein, beta amyloid precursor protein (β-APP or APP).

Importantly, the present inventors have shown that the polypeptides of the present invention bind specifically to IL-4Rα, and inhibit IL-4Rα binding to IL-13 and/or IL-4.

Therefore, in one embodiment, the polypeptide binds specifically to IL-4Rα and inhibits IL-4Rα binding to IL-13 and/or IL-4. In an embodiment, the polypeptide inhibits JAK-STAT signaling.

The term “IL4Rα” as used herein, refers to the alpha chain of the interleukin-4 receptor (also known as Interleukin-4 receptor subunit alpha (e.g. UNIPROT P24394; http://www.uniprot.org/uniprot/P24394), a transmembrane protein, which contains an extracellular domain of 207 amino acids. A secreted form of the extracellular domain exists, sIL-4R alpha, which is also known as CD124 and capable of blocking IL-4 activities. A polypeptide of the present invention may be able to bind sIL-4 receptor alpha as well as any portion of the extracellular domain of IL-4 receptor alpha.

Interleukin-4 (IL-4) is a T cell derived multifunctional cytokine that plays a critical role in the regulation of immune responses. IL-4 induces Th2 (T helper 2) differentiation, causes macrophage suppression, and stimulates B cell production of Immunoglobulins E.

IL-4 (and IL-13) signal via IL-4Rα, a component of the type I (IL-4Rα and a common gamma chain) and type II receptors (IL-4Rα and IL-13Rα1). IL-4 signals via both type I and II receptor pathways, whereas IL-13 signals only via the type II IL-4R. The common gamma chain activates Janus kinases (JAK) 1 and 3, whereas IL-13Rα1 activates tyrosine kinase 2 (TYK2) and JAK2. Activated JAKs then phosphorylate STAT-6. Phosphorylated STAT-6 dimerizes, migrates to the nucleus, and binds to the promoters of the IL-4 and IL-13 responsive genes, such as those associated with T-helper type 2 (Th2) cell differentiation, airway inflammation, airway hyper-responsiveness (AHR) and mucus production.

Without wishing to be bound by theory, IL-4 and IL-13 play key roles in Th2 immunity and the pathogenesis of atopic and allergic diseases. As discussed herein, the function of these cytokines is partially linked through their shared use of the IL-4Rα chain. The so-called Type I receptor comprising IL-4Rα and the common γ-chain, is expressed by hemopoietic cells and exclusively responds to IL-4, causing differentiation of naïve T cells into Th2 cells that are responsible for inducing IgE production by B cells. In contrast, the Type II receptor, comprising IL-4Rα and IL-13Rα1, is responsive to both IL-4 and IL-13. Upon binding with either IL-4 or IL-13, the cytoplasmic domain of IL-4Rα becomes tyrosine phosphorylated by the activated receptor-associated Janus kinases (JAKs), leading to the recruitment of STAT6, its subsequent phosphorylation, and formation into homodimers. These homodimers translocate to the nucleus, binding the promotors of responsive genes and activating the transcription of genes involved in B cell differentiation and immunoglobulin class switching. It is well established that STAT6 contributes to Ig class switching to produce IgE. Therefore, STAT6 is an important marker for the IL-4/IL-13 signaling pathway.

The term “JAK-STAT signaling” as used herein includes the downstream effects of STAT-6 phosphorylated by JAK1. For example, the promotion of transcription of target genes, including Suppressor of cytokine signaling 1 (SOCS1), IL4Rα, Chemokine (C-C motif) ligand 11 (Eotaxin), GATA binding protein 3 (GATA-3), Fc fragment of IgE, low affinity II, receptor for (CD23), Immunoglobulin heavy constant epsilon (IGHE), Immunoglobulin heavy constant gamma 1 (IGHG1) and Immunoglobulin heavy constant gamma 4 (IGHG4).

A role of IL-4 is the induction of IgE production by switching immunoglobulin production from IgM to IgE in B cells. IL-4 deficient mice show low IgE serum levels, which can be rescued by introduction of IL-4 positive bone marrow. Similarly, knockout of the downstream STAT6 results in a 100-fold decrease in IgE production and ablation of T cell development. Thus, IL-4Rα signaling via STAT6 plays an important physiological role, and represents an attractive therapeutic target, given its role as a common IL-4/IL-13 receptor.

“Polynucleotide,” or “nucleic acid,” as used interchangeably herein, refer to polymers of nucleotides of any length, and include, but are not limited to, DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl, 2′-fluoro- or 2′-azido-ribose, carbocyclic sugar analogs, alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(0)S(“thioate”), P(S)S (“dithioate”), “(0)NR.sub.2 (“amidate”), P(O)R, P(0)0R, CO or CH.sub.2 (“formacetal”), in which each R or R is independently H or substituted or unsubstituted alkyl (1-20 C.) optionally containing an ether (-0-) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

“Control sequences”, as used herein, are DNA sequences that enable the expression of an operably-linked coding sequence in a particular host organism. Prokaryotic control sequences include promoters, operator sequences, and ribosome binding sites. Eukaryotic control sequences include promoters, polyadenylation signals, and enhancers.

Nucleic acid is “operably-linked” when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “fusion protein” refers to a polypeptide having two portions covalently linked together, where each of the portions is derived from different proteins. The two portions may be linked directly by a single peptide bond or through a peptide linker containing one or more amino acid residues. Generally, the two portions and the linker will be in reading frame with each other and are produced using recombinant techniques.

The identification and characterization of the polypeptides that bind specifically to BACE1 and IL-4Rα as described herein provides compositions and methods for modulating the in vivo interactions between BACE1 and its substrates, e.g., APP, or IL-4Rα and its interacting partners, e.g. IL-4 and/or IL-13. The polypeptides that bind specifically to BACE1 and/or IL-4Rα as described herein may therefore be used in the treatment of diseases and disorders discussed below.

A “disorder” or “pathological condition” is any condition that would benefit from treatment with a substance/molecule or method of the invention. This includes chronic and acute disorders or diseases including those pathological conditions that predispose the mammal to the disorder in question.

Non-limiting examples of disorders to be treated herein include neurological disorders and disorders associated with an increase of the Th2 immune response.

The terms “neurological disorder” or “neurological disease” refer to or describe a disease or disorder of the central and/or peripheral nervous system in mammals. Examples of neurological disorders include, but are not limited to the following list of disease and disorders. Amyloidoses are a group of diseases and disorders associated with extracellular proteinaceous deposits in the CNS, including, but not limited to, secondary amyloidosis, age-related amyloidosis, Alzheimer's Disease (AD), mild cognitive impairment (MCI), Lewy body dementia, Down's syndrome, hereditary cerebral hemorrhage with amyloidosis (Dutch type); the Guam Parkinson-Dementia complex, cerebral amyloid angiopathy, Huntington's disease, progressive supranuclear palsy, multiple sclerosis; Creutzfeld Jacob disease, Parkinson's disease, transmissible spongiform encephalopathy, HIV-related dementia, amyotropic lateral sclerosis (ALS), inclusion-body myositis (IBM), and ocular diseases relating to beta-amyloid deposition (i.e., macular degeneration, drusen-related optic neuropathy, and cataract).

In an aspect, the present invention provides a method of treating an individual having a disorder associated with an increase of the Th2 immune response, said method comprising administering to the individual an effective amount of a polypeptide described herein.

The term “disorders associated with an increase of the Th2 immune response” refers to or describes a disease or disorder associated with an increase of the Th2 immune response. Th2-type immune responses promote antibody production and humoral immunity, and are elaborated to fight off extracellular pathogens. Th2 cells are mediators of Ig production (humoral immunity) and produce IL-4, IL-5, IL-6, IL-9, IL-10 and IL-13 (Tanaka, et. al., Cytokine Regulation of Humoral Immunity, 251-272, Snapper, ed., John Wiley and Sons, New York (1996)). Th2-type immune responses are characterized by the generation of certain cytokines (e.g., IL-4, IL-13) and specific types of antibodies (IgE, IgG4) and are typical of allergic reactions, which may result in watery eyes and asthmatic symptoms, such as airway inflammation and contraction of airway muscle cells in the lungs.

In contrast to the approaches of the prior art, and without wishing to be bound by theory, inhibition of IL-4/IL-13 interaction with IL-4Rα, has the potential to provide treatment to all allergies, since it targets the down-regulation of the IL-4Rα signaling pathway resulting in reduced phosphorylation of STAT6 and therefore less IgE. For example, a range of techniques to down-regulate the interaction between IL-4 and IL-4Rα, have been employed, including monoclonal antibodies, antagonists and soluble receptors, although the efficacy of these approaches still remains to be proven. Synthetic peptides based on T cell epitopes of major allergens (e.g. from cat and house dust mite), have been successfully used as therapeutics in experimental models of allergy, which have led to evaluation of synthetic peptides for immunotherapy in clinical trials. Such clinical studies have shown that peptide therapy reduced sensitivity to the allergen and the allergen-specific proliferative and cytokine responses were downregulated. However, peptide therapy approaches are allergen-specific, which are only effective for the treatment of allergy on which the T cell epitope peptides are based on.

Furthermore, the most common approaches toward allergy treatment focus on allergen avoidance and pharmacotherapy to neutralize allergic symptoms. Pharmacotherapy exploits antagonistic drugs such as antihistamines, antileukotreines, corticosteroids, cromolyn, methylxanthines, β-antagonists, muscarinic antagonists and mast cell stabilisers to block the actions of allergic mediators or to circumvent the degranulation process. However, such therapies do not ameliorate all symptoms due to their inability to inhibit IgE production. Furthermore, such antagonistic drugs can promote immunosuppression or non-immunological effects. Allergic desensitization has been used to improve immunological tolerance in allergic individuals. For example, allergen specific immunotherapy (SIT) involves weekly vaccination (subcutaneous) of increased doses of specific allergens into the patient with the aim of modifying Th2 cells into Th1 cells and therefore induce production of IgG rather than IgE. This method has been recognized as an effective treatment for rhinitis and asthma. However, administration of increased amounts of allergens may cause excessive production of IgE and lead to therapy-induced anaphylaxis, which represents a major drawback of SIT.

Therefore, in one embodiment, the disorder is associated with an allergic reaction or an allergic inflammation.

Allergy is an abnormal, symptomatic overreaction by the immune system to innocuous environmental substances, known as allergens, which include dust mites, peanuts and grass pollen. Allergens are a type of antigen that trigger a complex immune response upon contact with the immune system. This is classified as Type I hypersensitivity because of the immediate and inflammatory immune response, characterised by the excessive production of immunoglobulin E (IgE) antibodies.

Allergens are proteins or chemical substances that originate from a variety of animal and plant sources and exist abundantly in the environment. In indoor environments, some of the most common allergens are dust mites, pet fur and dander, cockroach calyx, mould and wool. Outdoor allergens include, but are not limited to, stings from insects such as bees and wasps, pollen from grasses, weeds and trees and fungal spores. Allergens can also be found in many of the foods that humans consume including eggs, milk, legumes (e.g. peanuts), seafood, soy, tree nuts, and wheat (gluten). Many individuals are also known to be allergic to artificial substances such as perfumes, latex and medications including penicillin and anaesthetics. Unlike the perennial indoor and food allergens, the outdoor allergens are seasonal with high levels attainable, making avoidance very difficult. In Australia, grass pollens are the major seasonal allergens affecting up to 40% of the population. In the last 20 years, much global research effort has been directed in the characterisation and cloning of major grass pollen allergens to permit the identification of their B and T cell epitopes for use in the preparation of hypoallergenic mutants in therapy.

Therefore, in one aspect, the present invention provides a method of reducing IgE in a patient comprising in a patient comprising administering to the patient an effective amount of a polypeptide described herein.

In one embodiment, the peptide of the invention is capable of reducing IgE in a patient, for example by at least 20%, or at least 30%, or at least 35%, 40%, 45%, 50%, 55%, 60%, 65%, 70%, 75%, 80%, 85% or 90%.

An effective amount may be determined using in vitro and/or in vivo models of a disorder associated with an increase of the Th2 immune response. For example, animal models based on natural allergens such as house dust mite (HDM) provide a relevant insight into the allergen-induced mechanisms that underpin human allergic diseases. The symptoms of asthma result from a complex anatomical, structural and physiological interplay of the respiratory system that is not easily replicated in vitro or in small laboratory rodent models. Sheep have long been used as relevant models for human lung structure, development and disease. The present inventors have established an ovine model for human allergic asthma that uses HDM, a relevant human allergen, and allows for the investigation of acute responses to airway allergen exposure as well as changes in lung structure and function in response to chronic challenge. In this model, sheep that are responsive to HDM allergen typically display allergen-specific IgE responses, airway eosinophilia and mucus hypersecretion of the airways, and in the chronic condition features of airway wall remodeling and decline in lung function. Importantly, these features of experimental asthma in the sheep model closely parallel that seen in the human asthmatic condition. The value of using larger animal models in the context of human airways disease includes the ability to perform procedures in line with those in humans, such as segmental or aerosolised allergen challenges, bronchoalveolar lavage (BAL) and endobronchial biopsy sampling. It has been reported that ovine/bovine IL-4 stimulates the human TF-1 cell line, suggesting the cross-species usefulness and relevance of this sheep model.

In one embodiment, the patient is suffering from, or at risk of contracting, a disorder associated with an increase of the Th2 immune response. In another embodiment, the disorder is associated with an allergic reaction or an allergic inflammation.

Allergic diseases include allergic asthma, rhinitis, asthma, hay fever, atopic eczema, atopic dermatitis and celiac disease. Individuals with atopy have a strong hereditary predisposition to produce excessive IgE in response to common environmental allergens and have a tendency to possess one or more allergies. The combination of atopy and allergens may sometimes lead to severe hypersensitivity conditions such as anaphylaxis, in which allergen-induced release of inflammatory mediators from mast cells and basophils results in a systematic and catastrophic physiological reaction, which can prove fatal.

In another embodiment, the disorder is associated with a mucus production or a mucus secretion. In another embodiment, the disorder is selected from the group consisting of allergic inflammation, allergic asthma, obstructive pulmonary disease, or adult respiratory distress syndrome.

In addition, the disorder that is preferably treated, ameliorated or prevented by the methods of the present disclosure by applying the peptides as described herein, may be associated with allergic reaction or allergic inflammation.

In some preferred embodiments, the disorder may be allergic asthma, rhinitis, conjunctivitis or dermatitis.

Asthma is a complex, persistent, inflammatory disease characterized by airway hyper-responsiveness in association with airway inflammation. Studies suggest that regular use of high-dose inhaled corticosteroids and long-acting bronchodilators or omalizumab (a humanized monoclonal antibody that binds to immunoglobulin E and is often used as next-step therapy) may not be sufficient to provide asthma control in all patients, highlighting an important unmet need, Interleukin-4, interleukin-13, and the signal transducer and activator of transcription factor-6 are key components in the development of airway inflammation, mucus production, and airway hyper-responsiveness in asthma.

In one embodiment, the allergic asthma is an airway inflammation in which the IL4/IL13 pathway contributes to disease pathogenesis.

Furthermore, the disorder that is preferably treated, ameliorated or prevented by the methods of the present disclosure by applying the polypeptides described herein, may also be lung disorders, for example, pulmonary disorders in which the IL4/IL13 pathway contributes to disease pathogenesis. Such pulmonary disorders include but are not limited to, lung fibrosis, including chronic fibrotic lung disease, other conditions characterized by IL-4-induced fibroblast proliferation or collagen accumulation in the lungs, pulmonary conditions in which a Th2 immune response plays a role, conditions characterized by decreased barrier function in the lung (e.g., resulting from IL-4-induced damage to the epithelium), or conditions in which IL-4 plays a role in an inflammatory response.

For example, Cystic fibrosis (CF) is characterized by the overproduction of mucus and development of chronic infections. Inhibiting IL-4Rα and the Th2 response will reduce mucus production and help control infections such as allergic bronchopulmonary aspergillosis (ABPA). Allergic bronchopulmonary mycosis occurs primarily in patients with cystic fibrosis or asthma, where a Th2 immune response is dominant. Inhibiting IL-4RA and the Th2 response will help clear and control these infections.

Similarly, chronic obstructive pulmonary disease (COPD) is associated with mucus hypersecretion and fibrosis. Inhibiting IL-4RA and the Th2 response will reduce the production of mucus and the development of fibrous thereby improving respiratory function and delaying disease progression. Bleomycin-induced pneumopathy and fibrosis, and radiation-induced pulmonary fibrosis are disorders characterized by fibrosis of the lung which is manifested by the influx of Th2, CD4+ cells and macrophages, which produce IL-4 and IL-13 which in turn mediates the development of fibrosis. Inhibiting IL-4RA and the Th2 response will reduce or prevent the development of these disorders.

Moreover, IL-4 and IL-13 induce the differentiation of lung epithelial cells into mucus-producing goblet cells. IL-4 and IL-13 may therefore contribute to an enhanced production of mucus in subpopulations or some situations. Mucus production and secretion contributes to disease pathogenesis in chronic obstructive pulmonary disease (COPD) and cystic fibrosis (CF). Thus, the disorder, associated with a mucus production or a mucus secretion (for example, overproduction or hypersecretion), can be preferably treated, ameliorated or prevented by the methods of the present disclosure by applying a polypeptide as described herein. In some preferred embodiments, the disorder, associated with a mucus production or a mucus secretion is preferably a chronic obstructive pulmonary disease (COPD) or a cystic fibrosis (CF). In other preferred embodiments, the composition of the disclosure further comprises an anti-mucus medicament.

Pulmonary alveolar proteinosis is characterized by the disruption of surfactant clearance. IL-4 increases surfactant product. In some further embodiments, use of an IL-4Rα antagonist such as a polypeptide of the disclosure to decrease surfactant production and decrease the need for whole lung lavage, is also contemplated herein.

Adult respiratory distress syndrome (ARDS) may be attributable to a number of factors, one of which is exposure to toxic chemicals. Therefore, as a preferred but non-limiting example, one patient population susceptible to ARDS is critically ill patients who go on ventilators, as ARDS is a frequent complication in such patients. In some further embodiments, an IL-4Rα antagonist such as a polypeptide of the disclosure may thus be used to alleviate, prevent or treat ARDS by reducing inflammation and adhesion molecules.

Sarcoidosis is characterized by granulomatous lesions. In some further embodiments, use of an IL-4Rα antagonist such as a polypeptide of the disclosure to treat sarcoidosis, particularly pulmonary sarcoidosis, is also contemplated herein.

Conditions in which IL-4-induced barrier disruption plays a role (e.g., conditions characterized by decreased epithelial barrier function in the lung) may be treated with IL-4Rα antagonist(s). Damage to the epithelial barrier in the lungs may be induced by IL-4 and/or IL-13 directly or indirectly. The epithelium in the lung functions as a selective barrier that prevents contents of the lung lumen from entering the submucosa. A damaged or “leaky” barrier allows antigens to cross the barrier, which in turn elicits an immune response that may cause further damage to lung tissue. Such an immune response may include recruitment of eosinophils or mast cells, for example. An IL-4Rα antagonist may be administered to inhibit such undesirable stimulation of an immune response.

Therefore, an IL-4Rα antagonist such as a polypeptide of the disclosure may be employed to promote healing of lung epithelium, in asthmatics for example, thus restoring barrier function, or alternatively, administered for prophylactic purposes, to prevent IL-4 and/or IL-13-induced damage to lung epithelium.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, peptides of the invention are used to delay development of a disease or disorder.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g. mice and rats). In certain embodiments, the individual or subject is a human.

An “effective amount” refers to an amount effective, at dosages and for periods of time necessary, to achieve the desired therapeutic or prophylactic result. A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms are dictated by, and directly dependent on, the unique characteristics of the active compound and the particular desired therapeutic effect, and the inherent limitations of compounding the active compound.

Peptides or polypeptides of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The peptide or polypeptide need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

In one aspect, the present invention provides a polypeptide as described herein for use as a medicament.

In another aspect, the present invention provides a polypeptide as described herein for use in treating Alzheimer's disease (AD). In one embodiment, the polypeptide as described herein is for use in decreasing and/or inhibiting amyloid-β (Aβ) protein production.

In an aspect, the present invention provides a use of the polypeptide described herein in the manufacture of a medicament. In one embodiment, the medicament is for the treatment of Alzheimer's disease (AD). In another embodiment, the medicament is for reducing and/or inhibiting amyloid-β (Aβ) protein production.

The peptide of the invention is capable of reducing amyloid-β (Aβ) protein production, for example by at least 20%, or at least 30%, or at least 35%, 40%, 45%, 50%, 55%, 60%, 65%, 70%, 75%, 80%, 85% or 90%.

In an aspect, the present invention provides a polypeptide as described herein for use in treating a disorder selected from the group consisting of allergic inflammation, allergic asthma, obstructive pulmonary disease, or adult respiratory distress syndrome.

In one aspect, the present invention provides a polypeptide as described herein for use in decreasing and/or inhibiting JAK-STAT signaling.

In one embodiment, the peptide of the invention is capable of decreasing and/or inhibiting JAK-STAT signaling, for example by at least 20%, or at least 30%, or at least 35%, 40%, 45%, 50%, 55%, 60%, 65%, 70%, 75%, 80%, 85% or 90%.

In one aspect, the present invention provides a use of the polypeptide as described herein in the manufacture of a medicament. In one embodiment, the medicament is for the treatment of a disorder selected from the group consisting of allergic inflammation, allergic asthma, obstructive pulmonary disease, or adult respiratory distress syndrome.

In one embodiment, the medicament is for reducing and/or inhibiting JAK-STAT signaling.

In an aspect, the present invention provides an isolated polypeptide comprising an amino acid sequence that competes with the polypeptide described herein for binding to BACE1 and/or IL-4Rα.

In an aspect, the present invention provides an isolated polypeptide as described herein, wherein the polypeptide is conjugated or fused to a cytotoxic agent, an amino acid sequence tag that enhances cell entry, or an amino acid sequence of a protein that normally undergoes absorptive mediated transcytosis or receptor mediated transcytosis through the blood-brain-barrier.

In an aspect, the present invention provides an isolated polypeptide as described herein, wherein the polypeptide is formulated for administration to the lung.

In one aspect, a polypeptide that binds specifically to BACE1 and/or IL-4Rα for use as a medicament is provided.

Peptides or polypeptides of the invention can be used either alone or in combination with other agents in a therapy. For instance, a peptide or polypeptide of the invention may be co-administered with at least one additional therapeutic agent.

For the prevention or treatment of disease, the appropriate dosage of a peptide or polypeptide of the invention (when used alone or in combination with one or more other additional therapeutic agents) will depend on the type of disease to be treated, the severity and course of the disease, whether the peptide or polypeptide is administered for preventive or therapeutic purposes, previous therapy, the patient's clinical history and response to the peptide or polypeptide, and the discretion of the attending physician. The peptide or polypeptide is suitably administered to the patient at one time or over a series of treatments. Depending on the type and severity of the disease, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

However, the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. The progress of this therapy is easily monitored by conventional techniques and assays.

The term “pharmaceutical formulation” refers to a preparation which is in such form as to permit the biological activity of an active ingredient contained therein to be effective, and which contains no additional components which are unacceptably toxic to a subject to which the formulation would be administered.

A peptide or polypeptide of the invention can be incorporated into compositions, which in some embodiments are suitable for pharmaceutical use. Such compositions typically comprise the peptide or polypeptide, and an acceptable carrier, for example one that is pharmaceutically acceptable. A “pharmaceutically acceptable carrier” includes any and all solvents, dispersion media, coatings, antibacterial and antifungal agents, isotonic and absorption delaying agents, and the like, compatible with pharmaceutical administration. Examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

A pharmaceutical composition is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents; for example, parabens, chlorobutanol, phenol, ascorbic acid, and thimerosal, can contain microorganism contamination. Isotonic agents; for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound (e.g., any modulator substance/molecule of the invention) in the required amount in an appropriate solvent with one or a combination of ingredients as required, followed by sterilization.

Generally, dispersions are prepared by incorporating the active compound into a sterile vehicle that contains a basic dispersion medium, and the other required ingredients. Sterile powders for the preparation of sterile injectable solutions, methods of preparation include vacuum drying and freeze-drying that yield a powder containing the active ingredient and any desired ingredient from sterile solutions.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

For administration by inhalation, the compounds are delivered as an aerosol spray from a nebulizer or a pressurized container that contains a suitable propellant, e.g., a gas such as carbon dioxide.

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

The term “cytotoxic agent” as used herein refers to a substance that inhibits or prevents a cellular function and/or causes cell death or destruction. Cytotoxic agents include, but are not limited to, radioactive isotopes (e.g. At21 1, 1131, 1125, Y90, Re186, Re188, Sm153, Bi212, P32, Pb212 and radioactive isotopes of Lu); chemotherapeutic agents or drugs (e.g. methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The invention will now be further described by way of Examples, which are meant to serve to assist one of ordinary skill in the art in carrying out the invention and are not intended in any way to limit the scope of the invention.

### EXAMPLES

### Example 1: BACE1 Direct Binding Assay with a Biotinylated Synthetic Peptide Antagonist

FIG. 1 shows the mean absorbance of light of the developed assay, which directly correlates to the amount of biotinylated peptide binding to the target (BACE1), immobilised on a 96-well plate. Different concentrations of peptide were used to test for binding to the target. The negative control did not contain any peptide and represents the background binding of the streptavidin-HRP conjugate, represented by the 0 nM concentration. The 0.1 nM concentration is significantly (*) different to the negative control. The error bars represent the confidence in the mean absorbance values (P<0.05). This example shows a Biotinylated peptide antagonist of BACE1 demonstrates an isolated polypeptide that binds specifically to BACE1.

### Example 2: BACE1 Inhibition Assay Based on the Cleavage of Fluorogenic APP Substrate with an Unbiotinylated Synthetic Peptide Antagonist

FIG. 2 shows the negative control (0 nM) does not contain any peptide and represents the upper limit of the BACE1 enzyme activity by mean fluorescent units on the y-axis. The peptide concentrations of 100 nM and 200 nM show a significant (*) difference to the upper limit when compared with the negative control. The mean fluorescent units are a representation of the cleaved product of APP by BACE1 (P<0.0 5). BACE1 peptide antagonist demonstrates more than 40% inhibition of BACE1 activity (indicated by vertical arrows). This example shows an isolated polypeptide that binds specifically to BACE1 inhibits a BACE1 enzyme activity.

### Example 3. Peptide Inhibition of Endogenous BACE1 from Cultured M17 Human Neuroblastoma Cells

The M17 cell lysate was subjected to BACE1 enzyme activity assay and was read on a fluorometer in duplicate with one experimental group of a positive control, without the peptide inhibitor (0 nM), and an experimental group, with an added 200 nM concentration of the peptide inhibitor. In FIG. 3, the cell lysate sample is displayed on the x-axis, with and without the peptide inhibitor. The mean fluorescent units are a representation of the cleaved product of APP by BACE1 (P<0.0 5). This data indicates the production of BACE1 in cultured human neuronal cells and the usefulness of this cell line in this study. Moreover, BACE1 peptide antagonist demonstrates more than 50% inhibition of endogenous BACE1 activity (indicated by the vertical arrow). This example shows an isolated polypeptide that binds specifically to BACE1 inhibits endogenous BACE1 enzyme activity.

### Example 4: Proteomic Analysis of Cultured M17 Human Neuronal Cells

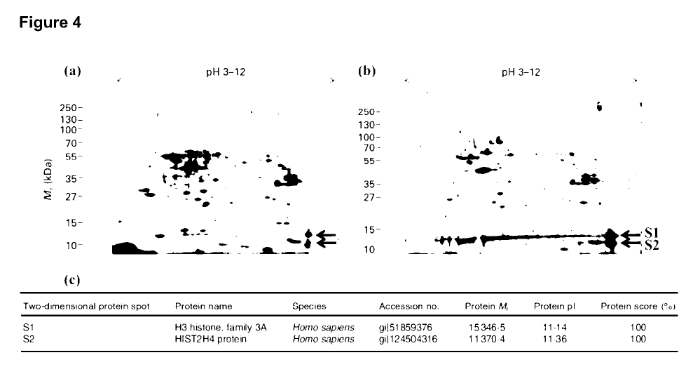

FIG. 4 shows the effect of DHA on protein expression in M17 human neuronal cells. Proteome expression of M17 cells grown in the presence of zinc (final concentration of 5 μM) and no DHA (a) or with 10 μg/mL DHA (b). Gels were stained with SYPRO Ruby staining. Protein spots of significant difference (arrows) were subjected to MS analysis (S1, S2). (c) Protein spots S1 and S2 were excised from each gel by automated robotic cutter and subjected to trypsin digest followed by MS analysis and submission of peptide fingerprints to Homo sapiens National Center for Biotechnology Information database searches. Proteins were identified via their peptide mass fingerprint and deduced amino acid sequence determined by single MS and tandem MS/MS, respectively. Protein identity was only reported for samples that gave a significant (P<0.05) molecular weight search score. pI, isoelectric point; Mr, molecular mass. This proteomic technique is to determine the cellular effect of the optimized BACE1 inhibitor analogue in cultured human neuronal cells.

### Example 5: Dose-Dependent Inhibition of Aβ Secretion from CHO-APP Cells Treated with a BACE1 Inhibitor

FIG. 5 shows a dose effect of the commercial BACE1 inhibitor, Compound IV (Merck Calbiochem) on Aβ release from a cellular model. Cells were treated for 5 hrs with compound concentrations ranging from 1 nM to 1 μM. Aβ secreted in the cell media was quantified using a sandwich ELISA. Data are calculated relative to Aβ secreted from control cells treated with DMSO (vehicle) alone. This data indicates the CHO-APP cell line described herein is useful as an in vitro model system to monitor the effectiveness of BACE1 inhibitor analogues and Compound IV as an inhibitor reference.

### Example 6: IL-4Rα Direct Binding Assay with a Biotinylated Synthetic Peptide Antagonist

FIG. 6 shows the mean absorbance of light of the developed assay, which directly correlates to the amount of biotinylated peptide binding to the target (IL-4Rα), immobilised on a 96-well plate. Different concentrations of peptide were used to test for binding to the target. The error bars represent the confidence in the mean absorbance values (P<0.05). This example shows a Biotinylated peptide antagonist of IL-4Rα demonstrates interaction with IL-4Rα. This data also shows a peptide antagonist specifically binds to biologically active human IL-4Rα. Importantly, the peptide antagonist could also interact with IL-4Rα in a dose-dependent manner, compared with a relevant negative control.

This example shows an isolated polypeptide that binds specifically to IL-4Rα.

In order to establish whether this synthetic peptide showed any inhibition of IL-4/IL-13 binding to IL-4Rα, we performed inhibition ELISA. FIG. 7 shows a peptide antagonist that specifically binds to biologically active human IL-4Rα and demonstrates significant inhibition of interaction between IL-4/IL-13 and IL-4Rα.

Pre-incubation of IL-4Rα with nM amounts of the peptide reduced IL-13 binding to IL-4Rα by 50% and IL-4 binding by 25%, suggesting that this peptide antagonist holds great promise as a potent inhibitor of IL-4/IL-13 interaction with IL-4Rα.

This example shows an isolated polypeptide that binds specifically to IL-4Rα inhibits IL-4Rα binding to interaction between IL-4 and/or IL-13.

### Example 7: Analysis of the Biomolecular Interaction Between IL-4 and IL-4Rα

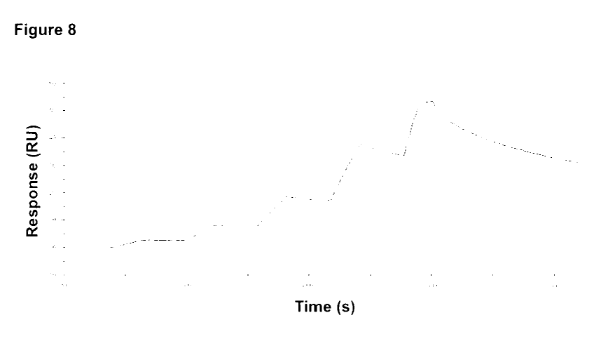

IL-4Rα was successfully immobilised on a CM-5 sensor chip and a single kinetic result for IL-4 interacting with IL4Rα. FIG. 8 shows a sensorgram of a kinetic titration analysis between IL-4 and IL-4Rα immobilized on a CM-5 sensor chip, as a solid curve fitting line. This data demonstrates an exceptional fit for 1:1 binding and fast association rate for IL-4. Biosensor technology is used to investigate both the capacity of the peptides to inhibit the interaction of IL-4/IL-13 with IL4Rα, but also the kinetics of the peptides with IL-4Rα, as compared with that of IL-4 and IL-13.

### Example 8: Inhibition of JAK-STAT Signaling

HEK-Blue cells are used to investigate the capacity of our peptide antagonist (and the generated peptide analogues) to inhibit IL-4/IL-13 signaling pathway in vitro.

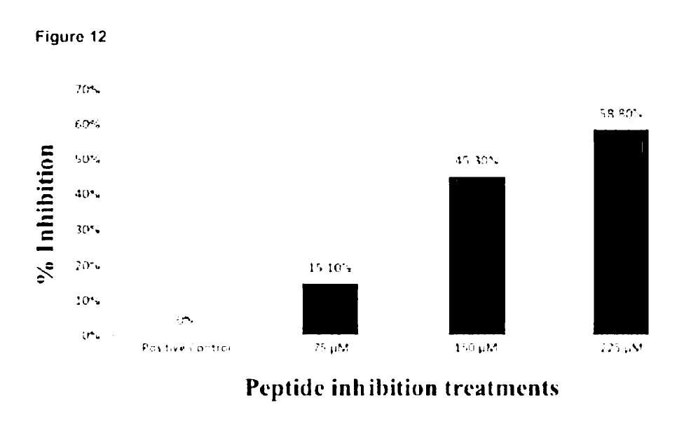

The peptide antagonist described herein demonstrates a dose-dependent inhibition of the JAK-STAT6 signaling in HEK-Blue cells, with up to 59% inhibition at 225 μM, as shown in FIGS. 9 and 12. The peptide antagonist itself (without the IL-4) did not trigger a non-specific JAK-STAT6 signaling, suggesting that this peptide antagonist is demonstrates specificity and efficacy.

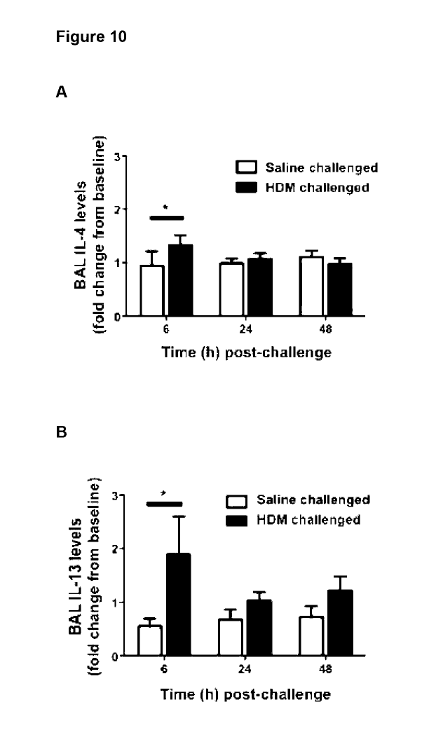

Recent studies using the sheep asthma model highlight the presence of IL-4 and IL-13 in BAL fluid following airway allergen challenge (FIG. 10), suggesting involvement of these key Th2 cytokines in the airway inflammation (recruitment of activated T cells, eosinophilia and IgE responses) typically seen in this animal model system.

### Example 9: Dissection of the Molecular Detail of the BACE1 Synthetic Peptide Inhibitor and Preparation of Peptide Analogues for Testing

Alanine scanning of the 12-mer BACE1 peptide antagonist is performed to determine amino acid (AA) residues that are critical for binding to BACE1 and inhibiting BACE1 enzyme activity, while truncated peptide analysis will determine the minimum peptide length required for the peptide's selectivity and efficacy.

Peptide analogues generated are tested in BACE1 enzyme assays and biosensor analyses using our well-established techniques.

Alanine scanning involves the sequential substitution of each AA residue of the original peptide with alanine to identify specific AA residues responsible for the peptide's activity. The degree of peptide activity reduction is taken as a relative measure of the importance of the AA being substituted. The present inventors have been previously successful in using such an alanine scanning technique to identify critical human IgE-binding AA residues of the major rye grass pollen allergen Lol p 5 (Suphioglu, C, Blaher, B, Rolland, J M, et al. (1998). Molecular basis of IgE-recognition of Lol p 5, a major allergen of rye-grass pollen. [symbol] Mol Immunology 35:293-305, incorporated herein by reference); the same techniques are used to identify AA residues of the 12-mer BACE1 peptide inhibitor that are critical for its activity.

For truncated peptide analysis, a truncated peptide library is constructed by systematically removing flanking AA residues of the original peptide from the N- and C-terminus, one AA residue at a time. Truncated peptide analysis is used to identify the shortest AA sequence needed for activity, as described in previous studies to determine the shortest peptide required for human IgE-binding of the major rye grass pollen allergen Lol p 5, above. Truncation of the original sequence of the 12-mer BACE1 peptide inhibitor one AA residue at a time, is used to determine the shortest peptide required for its binding and inhibition properties, and to assess effectiveness to cross the blood-brain barrier.

### Example 10: Peptide Synthesis

Peptides are produced commercially by Solid Phase Peptide Synthesis by the peptide supplier Auspep. The quality of peptide is assessed by HPLC and confirmed by mass spec (with greater than 90% purity).

Alanine scanning and truncated peptides are commercially by Auspep (Melbourne, Australia), an ISO 9002 certified company. 20-30 mg of each synthetic peptide with greater than 95% purity is obtained. Auspep have considerable experience with both Fmoc and Boc peptide chemistries, including the synthesis of difficult sequences. Auspep provides full analytical data, including HPLC and mass spectrometry for each peptide.

### Example 11: In Vitro Inhibition of BACE1 Activity by Optimized Peptide Analogues

Synthetic peptide analogues are used to test their affinity, selectivity and efficacy in BACE1 enzyme assays and biosensor analyses, and enzyme kinetic studies to define their mode of inhibition. To demonstrate that the optimized peptide antagonist is a selective BACE1 inhibitor, the following experiments are conducted:

BACE1 Enzyme Assays

A fluorescent BACE1 activity assay kit (Sigma-Aldrich, NSW, Australia), is used to not only show the cleavage of APP by BACE1, but also the inhibitors' ability to prevent cleavage of APP by BACE1. A measurement of fluorescence expresses the resulting cleaved APP product, which indirectly measures the activity of BACE1 in cleaving APP. Assays are conducted in triplicate with (1, 10, 100 and 1000 nM) or without the peptide inhibitors, as negative controls. Commercially available BACE1 inhibitors (STA-200 and Merck Compound IV) are used as references. Data derived from assays at various substrate concentrations are used to determine Ki values and the mode of inhibition (competitive/allosteric) using the Lineweaver-Burk method.

Assays for Specificity

An assay for BACE1 homologue, BACE2 is developed using the recombinant enzyme available from GenWay and the BACE1 substrate, as BACE2 was shown to cleave APP at bond-2,-1 of the β-secretase cleavage site.

Biosensor Analysis

Briefly, the interaction between BACE1 and the peptide inhibitor analogues is detected with a Biacore X-100 surface plasmon resonance biosensor (GE Healthcare Biosciences, VIC, AUS). BACE1 is immobilised to the sensor surface of a CM-5 sensor chip by the random amine coupling method and unliganded sites on the chip blocked with ethanolamine. Direct interaction of the peptide analogues with BACE1 is assessed by injection of different concentrations of the peptides onto the chip sequentially, without regeneration (kinetic titration). Kinetic parameters, such as ka and kd, are calculated with the Biacore evaluation software. Similarly, cathepsin D and BACE2 are be immobilized on individual CM-5 chips and subjected to the same analyses to further confirm the specificity of the synthetic peptide analogues.

Inhibition of BACE1 by Optimised Peptide Analogues in Cell Lines.

Two cell lines (M17 human neuronal and APP-transfected CHO) are used to investigate the in vitro inhibitory effect of the BACE1 peptide antagonist analogues on the BACE1 activity with APP cleavage and Aβ production and metabolism. The CHO-APP cells secrete amounts of Aβ that can be readily detected by direct western blotting or by ELISA. Other cell lines such as cells overexpressing BACE1 in the human neuroblastoma SHSY5Y line which produce detectable amounts of Aβ are used as additional cellular models.

Cell Culture

The present inventors have detected the presence of BACE1 in the cultured M17 human neuroblastoma cell line, which is extensively used and reported by our laboratory. M17 cells are grown at 37′C in a humidified atmosphere in the presence of 5.0% carbon dioxide, as monolayer cultures in 75 cm2 disposable plastic flasks (Nunc, Roskilde, Denmark), maintained in 10 mL of Opti-MEM media with heat inactivated 2.5% foetal bovine serum (FBS) supplementation. At ˜90% confluence, M17 cells are harvested or passaged using 0.025% Trypsin/EDTA or 0.05% trypsin/EDTA, respectively. Chinese hamster ovary (CHO) cells stably transfected with APP (CHO-APP) are also used. These are maintained in RPMI supplemented with 10% FBS. The SHSY5Y-BACE1 cells are maintained in DMEM/F12 (1/1) supplemented with 10% FBS, and 0.1 mM non-essential amino acids and 1 mM sodium pyruvate, and G418 geneticin (Invitrogen; selection antibiotic).

M17 Cell Treatments

M17 cells are seeded at a density of 1×10⁶ cells/75 cm2 flask and grown in media supplemented with (1, 10, 100 and 1000 nM) and without the peptide inhibitor analogues, as negative controls. After 24 and 48 hours of incubation, the cells are harvested, centrifuged at 1000×g for 5 mins and pellets resuspended in PBS. Each sample is then divided into aliquots, centrifuged at 14000×g for 5 mins and cell pellets stored at −80° C. until needed for analysis.

Cell Extraction

The CelLytic extraction kit (Sigma-Aldrich) is used to prepare total protein extracts from M17 cells, with protein concentrations determined by BCA protein assay (Thermo Scientific Pierce, NH, USA), following the manufacturer's instructions.

BACE1 Enzyme Assays:

Cell lysates obtained above are subjected to BACE1 enzyme assays, as described above.

CHO-APP Cell Treatments:

Cells are seeded in 12-well plates and treated for 6-24 hrs with peptide or vehicle. After treatment, the cells are examined under a light microscope to evaluate toxicity. Culture media is collected, centrifuged and stored at −20° C. for Aβ ELISA and for sAPPβ analysis. Cells are harvested in PBS and cell pellets collected by centrifugation and frozen at −80° C. until use. Pellets are homogenized in RIPA buffer (0.1% SDS, 0.5% sodium deoxycholate, 1% Triton X-100, 150 mM NaCl in 50 mM Tris-HCl, pH 7.4) to assay for BACE1 enzymatic activity. The data is normalised to protein concentration determined by the BCA.

Aβ ELISA

0.5 μg of mouse monoclonal antibody WO2 (Aβ1-16) is coated in 384-well plates. After blocking with hydrolysed casein and washing with PBS containing 0.05% Tween 20 (PBS-T), biotinylated IE8 antibody (Aβ18-22) (20 ng dilution in 10 μL blocking buffer) is added followed by culture media samples (50 μL/well). A standard curve of Aβ peptide (0.03-3 ng/well) is run in parallel. The plates are incubated overnight at 4° C. After washing with PBS-T, streptavidin-Europium solution (25 μL/well) is added to the plates, followed by incubation for 1 h at ambient temperature. Then, the plates are washed with PBS-T and the enhancement solution added (80 μL/well). The plates are read in a Wallac Victor 2 Multicounter instrument and data calculated relative to the Aβ peptide standard curve.

Analysis of Other APP Products

25 μg of cell lysates prepared as described above are denatured in Laemmli sample□buffer and electrophoresed on 8-12% Nu-PAGE gel (Invitrogen) and analysed by western blotting with APP C-terminal antibody (Merck) to detect C-terminal fragments (C99 and C83) as well as APP full length. Bands are visualized using a MicroChemi instrument (Berthold) and quantified with GelQuant software. Culture media is also tested in the Multiplex soluble APP assay that detects sAPPα and sAPPβ in the same well (Meso Scale Discovery).

### Example 12: Investigation of the Effect of the Optimised Peptide Analogue on Other Cellular Processes in a Human Neuronal Cell Line

To determine the effects of the optimised BACE1 peptide inhibitor on other cellular processes of the cultured M17 human neuronal cells, the treated cells described above are subjected to proteomic analysis using two-dimensional gel electrophoresis followed with mass spectrometric analysis of protein spots of interest, as shown in FIG. 4.

M17 cell pellets obtained as described above are resuspended in ZOOM protein solubilizer 1 lysis buffer (Invitrogen, CA, USA), disrupted by passing through a 21-gauge needle and sonicated by using a Microsone Ultrasonic cell disrupter (Misonix Incorporated, NY, USA), following our well-established techniques. Samples are then centrifuged at 14000×g for 20 mins at 4° C. and stored in small aliquots at −80° C. until needed for analysis. Protein concentrations of the cell lysates are determined by the BCA protein assay (Thermo Scientific Pierce). First (isoelectric focusing) dimension of reduced and alkylated cell lysates are resolved on pH 3-12 ZOOM IPG strips (Invitrogen) followed by the second dimension on precast 4-20% Tris-Glycine ZOOM gels (Invitrogen) following established protocols (Suphioglu, C, Sadli, N, Coonan, D, et al. (2010). Zinc and DHA have opposing effects on the expression levels of histones H3 and H4 [symbol] in human neuronal cells. Br J Nutrition 103:344-51). Gels will are stained with SYPRO Ruby protein gel stain (Invitrogen) and detected using an UV transilluminator to visualize the protein spots.

Comparison of 2D gels from M17 cells treated with and without the peptide inhibitor will reveal protein spots of differential expression, which are chosen for mass spectrometric analysis, following our well-established techniques (Suphioglu, C, Sadli, N, Coonan, D, et al. (2010). Zinc and DHA have opposing effects on the expression levels of histones H3 and H4 [symbol] in human neuronal cells. Br J Nutrition 103:344-51). A combined protein score for the peptide mass fingerprint and ten most intense peptides is obtained using the Mascot bioinformatic search engine (Department of Primary Industries, VIC, Australia) to search the NCBInr, Homo sapiens database, mass tolerance 100 ppm and oxidation as a variable modification. Proteins are identified via their peptide mass fingerprint and deduced amino acid sequence determined by single MS and tandem MS/MS, respectively.

### Example 13: Dissection of the Molecular Detail of the Human IL-4Rα Synthetic Peptide Antagonist and Preparation of Peptide Analogues for Testing

Alanine scanning of our 12-mer IL-4Rα peptide antagonist is performed to determine amino acid (AA) residues that are critical for not only binding to IL-4Rα but inhibiting IL-4/IL-13 binding to IL-4Rα. Truncated peptide analysis is performed to determine the minimum peptide length required for peptide activity. The peptide analogues generated in are tested in direct, inhibition and competition ELISAs and biosensor analyses, using our established techniques.

Alanine scanning involves the sequential substitution of each AA residue of the original peptide with alanine to identify specific AA residues responsible for the peptide's activity. The degree of peptide activity reduction is taken as a relative measure of the importance of the AA being substituted. The present inventors have been previously successful in using such an alanine scanning technique to identify critical human IgE-binding AA residues of the major rye grass pollen allergen Lol p 5 (Suphioglu, C, Blaher, B, Rolland, J M, et al. (1998). Molecular basis of IgE-recognition of Lol p 5, a major allergen of rye-grass pollen (Mol Immunology 35:293-305, incorporated herein by reference); the same techniques are used to identify AA residues of the 12-mer IL-4Rα peptide antagonist that are critical for its binding to IL-4Rα and inhibition of IL-4/IL-13 binding to IL-4Rα.

For truncated peptide analysis, a truncated peptide library is constructed by systematically removing flanking AA residues of the original peptide from the N- and C-terminus, one AA residue at a time. Truncated peptide analysis is used to identify the shortest AA sequence needed for activity, as described in previous studies to determine the shortest peptide required for human IgE-binding of the major rye grass pollen allergen Lol p 5, above. Truncation of the original sequence of the 12-mer IL-4Rα peptide antagonist is used to determine the shortest peptide required for its binding and inhibition properties.

Alanine scanning and truncated peptides are synthesised by Fmoc chemistry using manual Fmoc chemistry on PEG polystyrene resin. Couplings are carried out using HBTU and HOBt coupling reagents. Peptide purity is established using HPLC and identity confirmed by mass spectrometry and amino acid analysis.

### Example 14: Immunological Analyses

Alanine substituted and truncated peptide analogues are tested for functionality using our optimised in vitro immunoassays, as described herein.

Direct ELISA

Biologically active, purified human IL-4Rα (SBH Sciences, MA, USA) at 10 nM, is immobilised onto 96-well ELISA plates in triplicate, using established techniques. After extensive washing and blocking, plates are incubated with and without different concentrations (1-50 nM) of biotinylated peptides (including biotin as a negative control) for 1 hr at room temperature and washed again. Peptide binding is detected directly with horseradish peroxidase (HRP) conjugated streptavidin (Invitrogen, CA, USA), following the manufacturer's instructions. After extensive washing, HRP is colorimetrically detected with the HRP substrate o-phenylenediamine (Sigma-Aldrich, NSW, AUS) and read at 492 nm.

Inhibition ELISA

The same approach as described for direct ELISA above, is performed, with the exception that immobilised IL-4Rα is pre-incubated with and without different concentrations (1-50 nM) of unbiotinylated peptides (as the inhibitors) prior to incubation with 10 nM of either IL-4 or IL-13 (Sigma-Aldrich). The amount of IL-4/IL-13 binding is detected by incubation with the anti-IL-4 and anti-IL-13 murine monoclonal primary antibodies (Sigma-Aldrich), followed by HRP-conjugated anti-mouse secondary antibodies (Sigma-Aldrich). After extensive washing, HRP will be colorimetrically detected with the substrate o-phenylenediamine (Sigma-Aldrich) and read at 492 nm, according to our established protocols.

Competition ELISA

The same approach as described for inhibition ELISA described above, is performed with the exception that immobilised IL-4Rα is incubated with and without different concentrations (1-50 nM) of the peptides and either of IL-4 or IL-13, added at the same time. Cytokine binding is detected using the same approach as described above.

Biosensor Analysis

Briefly, the interaction between IL-4/IL-13 with IL-4Rα is detected with a Biacore surface plasmon resonance biosensor (GE Healthcare Biosciences, VIC, AUS). IL-4Rα is immobilised to the sensor surface of a CM-5 sensor chip by the random amine coupling method and unbound/reactive sites on the chip blocked with ethanolamine, following the manufacturer's instructions. The cytokine-receptor interaction experiments are conducted either with or without pre-incubation of the chip-immobilised IL-4Rα with the different concentrations of the peptide analogues, to assess the inhibitory capacity of the peptides. Direct interaction of the peptide analogues with IL-4Rα is assessed by injection of different concentrations of the peptides onto the chip sequentially, without regeneration (kinetic titration). Kinetic parameters, such as ka and kd, are calculated with the Biacore evaluation software.

### Example 15: In Vitro Inhibition of IL-4/IL-13/IL-4Rα Interaction by Optimised Peptide Analogues

The HEK-Blue cell line is used to investigate the in vitro inhibitory effect of IL-4Rα peptide antagonist analogues on the JAK/STAT6 signaling pathway.

Cell Line and Culture Conditions

HEK-Blue IL-4/IL-13 cells (InvivoGen, CA, USA) have been specifically designed to monitor the activation of the STAT6 pathway induced by IL-4 or IL-13, being stably transfected with STAT6 and the reporter gene secreted embryonic alkaline phosphatase (SEAP). Activation of this pathway leads to SEAP secretion in the supernatant and is easily detected using QUANTI-Blue medium that changes colour to purple/blue in the presence of SEAP, following manufacturer's instructions. Cells are grown and maintained in DMEM medium with 10% FCS at 37° C. in a 5% _{CO2}-humidified chamber, following the manufacturer's instructions and established cell culture techniques. These HEK-Blue cells are utilized in Example 8, which demonstrates up to 59% inhibition of the JAK/STAT6 signaling with the peptide antagonist of the present invention.

Cytokine, Peptide and Neutralising Antibody Treatments

The conditions for maximal STAT6 phosphorylation by performing kinetic studies are optimized using HEK-Blue cells (50,000 cells/well of a 96-well plate) either left untreated (“cells only” control) or treated in triplicate with 10 ng/mL of either IL-4, IL-13 or TNF-α (negative control; cell line does not respond to TNF-α) and grown for 0, 0.5, 1, 2, 4, 6, 8 or 24 hrs. At indicated time points, cells are harvested and lysed for Western blot analysis of total and phosphorylated STAT6 (see below). In addition, the supernatant of induced HEK-Blue cells is used for the SEAP reporter gene expression. Once optimised, the IL-4/IL-13 induced STAT6 activation pathway of the cell line is examined in the presence and absence of different concentrations (0-225 uM) of the original IL-4Rα peptide antagonist and analogues generated as described herein. Peptide treatment experiments involve either pre-incubation of the cells with peptides for 1 hr prior to the cytokine induction (to assess their prophylactic properties) or added at the same time as the cytokines (to test their competitive properties with the cytokines). Anti-IL-4/IL-13 neutralising antibodies (InvivoGen) are used as positive controls.

In FIG. 9, six treatment wells were used in total; 4 wells treated with peptide N1 and 2 wells without peptide as positive and negative controls (shown in results section). Cells in the treatment wells were incubated with 50 μl of N1 peptide (75, 150 and 225 μM, respectively) at 37° C. in a rotator shaker for 1 h, whereas the control wells were incubated with filter-sterilized water (DH2O). Post-incubation, 20 μl of IL-4 cytokine (100 ng/ml) was added to the positive control and three treatment wells. The 96-well plate was sealed using a parafilm and incubated at 37′C with 5% CO₂ for a period of 24 hours. Post-incubation, QUANTI-Blue substrate was prepared using the instructions in the HEK-Blue kit and 180 μl of this solution was added to 6 wells in a fresh 96-well plate. 20 μl of induced HEK-Blue IL-4 cells supernatant from each of the treatment wells was added to the QUANTI-Blue solution. The subsequent results were read using an xMark microplate absorbance spectrophotometer (Bio-Rad) at a wavelength of 640 nm

Cell Extraction and Western Blot Analysis

The CelLytic NuClear extraction kit (Sigma-Aldrich) is used to prepare nuclear and cytoplasmic protein extracts from HEK-Blue cells, with protein concentrations determined by BCA protein assay (Thermo Scientific Pierce, NH, USA), following the manufacturer's instructions. Cell lysates are analysed by SDS-PAGE and Western blotting, following our established techniques. Anti-STAT6 or anti-phospho-STAT6(Y641) primary rabbit polyclonal antibodies (Abcam, MA, USA) are used to detect total and phosphorylated STAT6 levels, respectively, following manufacturer's instructions and detected with HRP-conjugated anti-rabbit secondary antibodies (Chemicon, CA, USA), following our standard techniques. The same membranes are also probed for β-actin to ensure equal protein loading and facilitate densitometric analysis, following established techniques.

### Example 16: In Vivo Inhibition of IL-4/IL-13/IL-4Rα Interaction by Optimised Peptide Analogues

The in vivo inhibition of the IL-4Rα signaling pathway by the original peptide antagonist of IL-4Rα and its key peptide analogues is tested in an established sheep model of allergic asthma. The disease features of this model have been extensively characterised shown to closely resemble those of human asthma, making it an ideal experimental model for the human disease (Meeusen E N, et al. (2010). Drug Discovery Today: Disease Models 6:101-6, incorporated herein by reference). In addition, it has been reported that ovine/bovine IL-4 stimulates the human TF-1 cell line, suggesting the relevance of this sheep model to human disease.

Sheep Model Setup

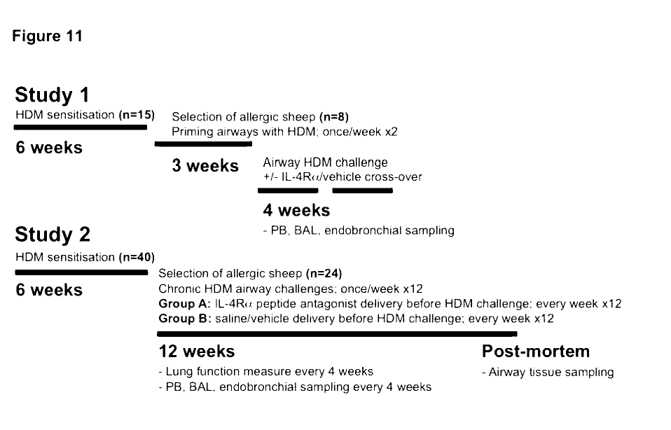

Sheep are sensitised to allergen by subcutaneous injections; approximately 50% of sheep respond to the sensitisation protocol (i.e. become ‘allergic’) as defined by increases in plasma HDM-specific IgE levels. Sheep classed as allergic are used to investigate the effect of inhibition of the IL-4Rα signaling pathway in vivo in the acute and chronic forms of the model. The experimental protocol used is outlined in FIG. 11.

Study 1 focuses on the acute phase of the sheep asthma model, established in allergic (sensitized) sheep following 2-3 airway challenges with HDM and displays key features of allergic airway inflammation.

Study 2 focuses attention on the chronic aspects of the sheep asthma model, established by repeated (weekly) airway allergen challenges over a period of 12 weeks, a regime that has been shown to induce significant features of chronic allergic airway disease in this model.

### Example 17: In Vivo Testing of Key Peptide Analogues

In Study 1 and Study 2, key peptide analogues are administered to allergic sheep as an aerosol at 24 h and 1 h before airway HDM allergen challenge to block the activity of IL-4Rα.

Study 1 (n=8 allergic sheep; cross-over design) examines the acute responses to airway allergen challenge with/without prior administration of IL-4Rα peptide antagonist; the key measureable outcome is whether treatment can suppress the induction of asthmatic airway inflammation. Aerosolised peptide antagonists/vehicle preps are delivered into the lungs (0.5-1 mg/kg in 3 ml saline/10% propylene glycol) using a jet nebuliser attached to a ventilator for controlled whole lung delivery. Study 1 also compares the in vivo efficacy of peptide antagonist delivery via intravenous (IV) and inhalation (aerosolised) routes; the more effective route for IL-4Rα peptide antagonist delivery is used for Study 2 (see below).

Segmental lobe HDM and saline challenges (aerosolized) are given via a tip-based nebuliser-catheter directed into defined segments/lobes of the airways using a fibre-optic endoscope (bronchoscope). The bronchoscope enables entry into the well-separated major left caudal lobe for delivery of HDM allergen, while the right caudal lobe serves as an internal control and receive sterile saline alone.

Bronchoaveolar lavage (BAL) and endobronchial biopsy samples are collected from the relevant lung lobes prior to (−24 h, 0 h) and 24 h, 48 h and 7 days following segmental allergen/saline challenge. Peripheral blood (PB) samples are collected 24 h before and 7 days after allergen challenge. Sheep are administered either peptide antagonist or vehicle alone in a randomized cross-over design, with a 2 week rest period between treatments.

Study 2 (n=40 allergic sheep; 2 treatment groups) investigates the effects of sustained IL-4Rα peptide antagonist delivery on development of the chronic asthmatic condition in the sheep asthma model. Study 2 examines the effects on measures of airway wall remodelling and decline in lung function; features typically seen in chronic asthmatics. Allergic (sensitized) sheep used in Study 2 are randomly assigned to one of 2 groups; these groups are given aerosolised peptide antagonists (Group A) or a vehicle prep (Group B), and concurrent repeated weekly challenge inhalations of HDM allergen (whole lung delivery using a nebuliser) over a period of 12 weeks. Based on our previous studies using the sheep asthma model, a 12-week period of challenges is the time-frame required to observe a significant decline in lung function (i.e. increase in baseline RL and increased AHR) following chronic airway allergen exposure. BAL samples, endobronchial biopsies and PB collections (as detailed for Study 1) take place before and 48 h after each of the HDM challenges. Lung function measurements are performed before and throughout the 12-week period of HDM/saline challenges, as indicated in FIG. 11. At the end of the 12-week challenge period, animals are sacrificed and lung tissues collected for detailed histological/immunochemical studies to investigate degree of airway wall remodelling (see below).

Immune Analyses and Assessment of Airway Function

Airway inflammation is assessed in BAL samples by way of differential cell counts, phenotypic analysis of BAL cells by flow cytometry (leucocytes, T cell subsets, activation status, etc.), and cytokine analysis (including IL-4, IL-5, IL-6, IL-10, IL-12, IL-13, GM-CSF, TNFα, MCP-1) in BAL cells and BAL fluid at the message (real-time RT-PCR) and protein (ELISA, flow cytometry) levels. Allergen-specific IgE, IgA, IgG1 and IgG2 levels are determined by ELISA in BAL and PB samples.

Airway tissues from endobronchial biopsies and post-mortem lung tissues are processed for detailed histology and immunostaining of paraffin-embedded and frozen tissue sections (H&E, Masson's Trichrome, PAS/Alcian blue, inflammatory cell infiltration, epithelial cell morphology, reticular basement membrane thickening, airway smooth muscle content) and real-time RT-PCR analyses to evaluate the extent of airway tissue inflammation and remodelling following allergen exposure with/without prior administration of the IL-4Rα peptide antagonist. Airway function measurements (Study 2) is performed in fully conscious animals over the 12-week allergen challenge period, using techniques developed in our laboratory for the assessment of airway lung function (Koumoundouros E, et al. (2006). Exp Lung Res 32:321-30, and Snibson K J, et al. (2006). Exp Lung Res 32:215-28, both incorporated herein by reference). Specific lung responses to allergen are determined just prior to and following the allergen challenge (for up to 60 min). AHR to carbachol (i.e. non-specific airway responses) is determined by increasing the dose of aerosolised carbachol (0.25-4.0% w/v in saline) until there is a 100% increase in baseline RL; carbachol responses are measured 24 h before and 24 h after HDM challenge. The breath-by-breath analyses will be used to determine RL and dynamic compliance from an average of 5 breaths.

1. An isolated polypeptide that binds specifically to BACE1 and/or IL-4Rα, wherein the polypeptide comprises the amino acid sequence FHESWPTFLSPS (SEQ ID NO: 1) or a biologically active derivative thereof.

2. The polypeptide of claim 1, wherein the polypeptide binds specifically to BACE1 and inhibits BACE1 enzyme activity.

3. The polypeptide of claim 1 or claim 2, wherein the polypeptide inhibits endogenous BACE1 enzyme activity.

4. The polypeptide of any one of claims 1 to 3, wherein the polypeptide inhibits amyloid-β (Aβ) secretion from cells.

5. The polypeptide of claim 1, wherein the polypeptide binds specifically to IL-4Rα and inhibits IL-4Rα binding to IL-13 and/or IL-4.

6. The polypeptide of claim 1 or claim 5, wherein the polypeptide inhibits JAK-STAT signaling.

7. An isolated polynucleotide encoding the polypeptide of any of claims 1 to 6, or a complement thereof.

8. A vector comprising the polynucleotide of claim 7.

9. A host cell comprising the vector of claim 8.

10. A method of producing a polypeptide comprising culturing the host cell of claim 9 under conditions in which the polynucleotide is expressed.

11. An antibody that specifically binds to the polypeptide of any one of claims 1 to 6, or a fragment thereof.

12. A kit comprising a polypeptide of any one of claims 1 to 6.

13. A pharmaceutical formulation comprising the polypeptide of any one of claims 1 to 6 and a pharmaceutically acceptable carrier.

14. A method of treating an individual having a neurological disease or disorder, said method comprising administering to the individual an effective amount of the polypeptide of any one of claims 1 to 4.

15. A method of reducing amyloid plaques in a patient suffering from, or at risk of contracting, a neurological disease or disorder, said method comprising administering to the individual an effective amount of the polypeptide of any one of claims 1 to 4.

16. A method of inhibiting amyloid plaque formation in a patient suffering from, or at risk of developing, a neurological disease or disorder, said method comprising administering to the individual an effective amount of the polypeptide of any one of claims 1 to 4.

17. The method of any one of claims 14 to 16, wherein the neurological disease or disorder is Alzheimer's disease (AD).

18. A method of reducing amyloid-β (Aβ) protein in a patient comprising administering to the patient an effective amount of the polypeptide of any one of claims 1 to 4.

19. The method of claim 18 wherein the patient is suffering from, or at risk of contracting, a neurological disease or disorder.

20. The method of claim 19, wherein the neurological disease or disorder is Alzheimer's disease (AD).

21. A method of treating an individual having a disorder associated with an increase of the Th2 immune response, said method comprising administering to the individual an effective amount of the polypeptide of any one of claim 1, 5 or 6.

22. A method according to claim 21 wherein said disorder is associated with an allergic reaction or an allergic inflammation.

23. A method according to claim 21 or claim 22 wherein said disorder is associated with a mucus production or a mucus secretion.

24. The method according to claim 21 or claim 22 wherein said disorder is selected from the group consisting of allergic inflammation, allergic asthma, obstructive pulmonary disease, or adult respiratory distress syndrome.

25. A method of reducing IgE in a patient comprising in a patient comprising administering to the patient an effective amount of the polypeptide of any one of claim 1, 5 or 6.

26. The method of claim 25 wherein the patient is suffering from, or at risk of contracting, a disorder associated with an increase of the Th2 immune response.

27. A method according to claim 26 wherein said disorder is associated with an allergic reaction or an allergic inflammation.

28. A method according to claim 26 or claim 27 wherein said disorder is associated with a mucus production or a mucus secretion.

29. The method according to claim 26 or claim 27 wherein said disorder is selected from the group consisting of allergic inflammation, allergic asthma, obstructive pulmonary disease, or adult respiratory distress syndrome.

30. The polypeptide of any one of claims 1 to 6 for use as a medicament.

31. The polypeptide of any one of claims 1 to 4 for use in treating Alzheimer's disease (AD).

32. The polypeptide of any one of claims 1 to 4 for use in decreasing and/or inhibiting amyloid-β (Aβ) protein production.

33. Use of the polypeptide of any one of claims 1 to 6 in the manufacture of a medicament.

34. The use of claim 33, wherein the medicament is for the treatment of Alzheimer's disease (AD).

35. The use of claim 33, wherein the medicament is for reducing and/or inhibiting amyloid-β (Aβ) protein production.

36. The polypeptide of any one of claim 1, 5 or 6 for use in treating a disorder selected from the group consisting of allergic inflammation, allergic asthma, obstructive pulmonary disease, or adult respiratory distress syndrome.

37. The polypeptide of any one of claim 1, 5 or 6 for use in decreasing and/or inhibiting JAK-STAT signaling.

38. Use of the polypeptide of any one of claim 1, 5 or 6 in the manufacture of a medicament.

39. The use of claim 38, wherein the medicament is for the treatment of a disorder selected from the group consisting of allergic inflammation, allergic asthma, obstructive pulmonary disease, or adult respiratory distress syndrome.

40. The use of claim 33, wherein the medicament is for reducing and/or inhibiting JAK-STAT signaling.

41. An isolated polypeptide comprising an amino acid sequence that competes with the polypeptide of any of claims 1 to 6 for binding to BACE1 and/or IL-4Ra.

42. An isolated polypeptide of any one of claims 1 to 4, wherein the polypeptide is conjugated or fused to a cytotoxic agent, an amino acid sequence tag that enhances cell entry, or an amino acid sequence of a protein that normally undergoes absorptive mediated transcytosis or receptor mediated transcytosis through the blood-brain-barrier.

43. An isolated polypeptide of any one of claim 1, 5 or 6, wherein the polypeptide is formulated for administration to the lung.
